(19) United States
(12) Patent Application Publication
(10) Pub. No.: US 2025/0230149 A1
(43) Pub. Date: Jul. 17, 2025

(54) MGLUR5 MODULATING COMPOUNDS, COMPOSITIONS, AND METHODS OF USE

(71) Applicants: Allyx Therapeutics, Inc., New Haven, CT (US); Yale University, New Haven, CT (US)

(72) Inventors: Timothy Ryan SIEGERT, New Haven, CT (US); Stephen STRITTMATTER, New Haven, CT (US); Federico SCARAVELLI, Verona (IT); Camilla CORÁ, Verona (IT)

(21) Appl. No.: 18/849,063

(22) PCT Filed: Mar. 22, 2023
(86) PCT No.: PCT/US2023/015966

§ 371 Date: Sep. 20, 2024

Related U.S. Application Data
(60) Provisional application No. 63/322,446, filed on Mar. 22, 2022.

(51) Int. Cl. C07D 413/04; A61K 9/48; A61K 31/4439; A61P 25/28

(52) U.S. Cl. CPC C07D 413/04; A61K 9/4858; A61K 9/4866; A61K 31/4439; A61P 25/28

(57) ABSTRACT

The present disclosure provides compositions of (4R,5R)-5-(2-chlorophenyl)-4-(5-(phenylethynyl)pyridin-3-yl)oxazo-lidin-2-one (Compound 1). Crystalline and solvate forms of the compound and formulations comprising the compound are also provided. Methods of using the compound and methods of administering the formulations to a subject in need thereof are provided to treat or prevent CNS disorders such as Alzheimer's disease.

### STATEMENT REGARDING FEDERALLY SPONSORED RESEARCH OR DEVELOPMENT

This invention was made with government support under U01 AG058608 awarded by the the National Institutes of Health. The government has certain rights in the invention.

### BACKGROUND

Glutamate is the primary excitatory neurotransmitter in the brain and is involved in various psychiatric and medical conditions. Glutamate regulates central nervous system functions through the actions of ionotropic and metabotropic receptors. The three groups of metabotropic (mGlu) receptors modify neuronal activity through G-protein coupled signaling. In particular, the Group I receptor, mGluR5 receptor, plays an important role in mental health and various central nervous system (CNS) disorders. Several mGluR5 antagonists have been designed, such as 3-[2-methyl-1,3-thiazol-4yl]ethynyl]pyridine (MTEP), acamprostate, memantine, AFQ056, and Fenobam. Allosteric modulators of mGluR5 can be sorted into positive allosteric modulators (PAMs), negative allosteric modulators (NAMs), and silent allosteric modulators (SAMs).

There remains a need for compounds and compositions that modulate mGluR5 to improve mental health and treat CNS disorders.

### SUMMARY OF THE DISCLOSURE

In one aspect of the disclosure, a pharmaceutical composition including (4R,5R)-5-(2-chlorophenyl)-4-(5-(phenylethynyl)pyridin-3-yl)oxazolidin-2-one

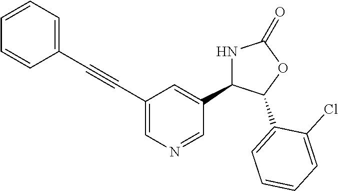 [SMILES: O=C1N[C@H](c2cncc(C#Cc3ccccc3)c2)[C@@H](c2ccccc2Cl)O1]

(Compound 1) is provided. In some embodiments, the pharmaceutical composition includes Compound 1, lactose monohydrate, croscarmellose sodium, and/or magnesium stearate.

Another aspect of the disclosure provides a method of treating a CNS disorder by administering a therapeutically effective amount of Compound 1, or a pharmaceutically acceptable salt thereof, to a subject in need thereof. In some embodiments, the CNS disorder is Alzheimer's disease. In some embodiments, a therapeutically effective amount of Compound 1, or a pharmaceutically acceptable salt thereof, is used for preventing seizure, reversing synapse loss, or reducing Tau accumulation in a subject in need thereof.

The disclosure further provides a crystalline form of Compound 1, e.g., in an anhydrous crystalline form. The disclosure further provides a solvate of Compound 1, e.g., an isopropyl solvate.

In an aspect, the invention features an anhydrous (AH) crystalline form (Form A) of Compound 1 having an XRPD pattern including peaks of 2θ angles at 9.02, 11.65, and 11.86 degrees 2θ, each ±0.2 degrees 2θ, as measured by X-ray diffractometry by irradiation with Cu Kα X-rays. In some embodiments, the XRPD pattern further includes peaks of 2θ angles at 12.15, 14.99, 28.99, and 44.03 degrees 2θ, each ±0.2 degrees 2θ, as measured by X-ray diffractometry by irradiation with Cu Kα X-rays. In some embodiments, the XRPD pattern further includes three peaks of 2θ angles at 21.09, 21.49, and 21.88 degrees 2θ, each ±0.2 degrees 2θ, as measured by X-ray diffractometry by irradiation with Cu Kα X-rays. In some embodiments, the XRPD pattern includes peaks of 2θ angles at 9.02, 11.65, 11.86, 12.15, 14.99, 21.09, 21.49, 21.88, 28.99, and 44.03 degrees 2θ, as measured by X-ray diffractometry by irradiation with Cu Kα X-rays. In some embodiments, the XRPD pattern is substantially as shown in FIG. 3.

In some embodiments, the AH form has a melting endothermic peak at about 134° C.-138° C. in a differential scanning calorimetry (DSC) thermogram. In some embodiments, the AH form has a DSC thermogram substantially as the DSC graph shown in FIG. 4. In some embodiments, the AH form has a thermogravimetric analysis (TGA) weight loss of about 0.02% (w/w) between ambient and about 250° C. In some embodiments, the AH form has a TGA substantially as the TGA graph shown in FIG. 4. In some embodiments, the AH form is substantially purified.

In an aspect, the invention features a method for preparing an anhydrous (AH) crystalline form (Form A) of Compound 1, including precipitating the anhydrous crystalline form from a solution including Compound 1 and a solvent selected from the group consisting of ethyl acetate (EtOAc), heptane, and mixtures thereof. In some embodiments, the solvent is a mixture of EtOAc and heptane. In some embodiments, the ratio of EtOAc and heptane is 90:10, 80:20, 70:30, 60:40, 50:50, 40:60, 30:70, 20:80 or 10:90. In some embodiments, the method further includes cooling the solution. In some embodiments, the method further includes isolating the AH crystalline form by filtration.

In an aspect, the invention features a solvate form (Form B) of Compound 1 having an XRPD pattern including at least three of the following peaks of 2θ angles at 16.07, 16.27, 16.58, and 16.77 degrees 2θ, each ±0.2 degrees 2θ, as measured by X-ray diffractometry by irradiation with Cu Kα X-rays. In some embodiments, the XRPD pattern further includes three or more of the following peaks of 2θ angles at 13.18, 13.4, 13.54, 13.54, and 13.70 degrees 2θ, each ±0.2 degrees 2θ, as measured by X-ray diffractometry by irradiation with Cu Kα X-rays. In some embodiments, the XRPD pattern further includes peaks of 2θ angles at 37.31, and 39.92 degrees 2θ, each ±0.2 degrees 2θ, as measured by X-ray diffractometry by irradiation with Cu Kα X-rays. In some embodiments, the XRPD pattern includes peaks of 2θ angles at 13.18, 13.4, 13.54, 13.54, 13.70, 16.07, 16.27, 16.58, 16.77, 37.31, and 39.92 degrees 2θ, each ±0.2 degrees 2θ, as measured by X-ray diffractometry by irradiation with Cu Kα X-rays. In some embodiments, the solvate Form B has an XRPD pattern substantially as shown in FIG. 5.

In some embodiments, the solvate Form B has a melting endothermic peak at about 134° C.-138° C. in a differential scanning calorimetry (DSC) thermogram. In some embodiments, the solvate Form B has a DSC thermogram substantially as the DSC graph shown in FIG. 6. In some embodiments, the solvate Form B has a thermogravimetric analysis (TGA) weight loss of about 6.7% (w/w) between ambient and about 200° C. In some embodiments, the solvate Form B has a TGA substantially as the TGA graph shown in FIG. 6. In some embodiments, the solvate Form B is an isopropyl alcohol solvate. In some embodiments, the solvate Form B is substantially purified.

In an aspect, the invention features a method for preparing solvate Form B of Compound 1, including the evaporation of a mixture of Compound 1 and isopropyl alcohol under inert atmosphere in a dry-box. In some embodiments, the evaporation occurs over a period of 1-2 weeks. In some embodiments, the method further includes drying the solvate Form B under vacuum.

In an aspect, the invention features a solvate form (Form C) of Compound 1, having an XRPD pattern including only two peaks between 13.32 and 13.82 degrees 2θ, each ±0.2 degrees 2θ, as measured by X-ray diffractometry by irradiation with Cu Kα X-rays. In some embodiments, the XRPD pattern further includes only three peaks between 2θ angles at 32.22 and 32.96 degrees 2θ, each ±0.2 degrees 2θ, or includes peaks of 2θ angles at 3.4, 8.33, 10.94, 16.32, and 16.66 degrees 2θ, each ±0.2 degrees 2θ as measured by X-ray diffractometry by irradiation with Cu Kα X-rays. In some embodiments, the XRPD pattern includes peaks of 2θ angles at 3.4, 8.33, 10.94, 13.32, 13.82, 16.32, 16.66, 32.22, 32.59, 32.96 degrees 2θ, each ±0.2 degrees 2θ, as measured by X-ray diffractometry by irradiation with Cu Kα X-rays. In some embodiments, the solvate Form C has an XRPD pattern substantially as shown in FIG. 7. In some embodiments, the solvate Form C has an XRPD pattern substantially as shown in FIG. 9.

In some embodiments, the solvate Form C has a melting endothermic peak at about 129-133° C. in a differential scanning calorimetry (DSC) thermogram. In some embodiments, the solvate Form C has a DSC thermogram substantially as the DSC graph shown in FIG. 8. In some embodiments, the solvate Form C has a thermogravimetric analysis (TGA) weight loss of about 3.4% (w/w) between ambient and about 200° C. In some embodiments, the solvate Form C has a TGA substantially as the TGA graph shown in FIG. 8. In some embodiments, Form C is an isopropyl alcohol solvate. In some embodiments, Form C is substantially purified.

In an aspect, the invention features a method for preparing solvate Form C of Compound 1 using a long-term slurry. In some embodiments, the method further includes the precipitation of the solvate form from a solution including Compound 1 and isopropyl alcohol. In some embodiments, the method further includes stirring the solution at 20° C. over two weeks. In some embodiments, the method further includes isolating the solvate Form C by filtration.

In some embodiments, the solvate Form C has endothermic peaks at about 89.4° C. and at about 134.2° C. in a differential scanning calorimetry (DSC) thermogram. In some embodiments, the solvate Form C has a DSC thermogram substantially as the DSC graph shown in FIG. 10. In some embodiments, the solvate Form C has a thermogravimetric analysis (TGA) weight loss of about 3.2% (w/w) between ambient and about 200° C. In some embodiments, the solvate Form C has a TGA substantially as the TGA graph shown in FIG. 10.

In an aspect, the invention features a method for preparing solvate Form C of Compound 1 using vapor diffusion. In some embodiments, the method further includes:
(i) dissolving Compound 1 in isopropyl alcohol to form a saturated solution;
(ii) filtering the mixture of step (i) into a vial that is placed in an outer vial containing an anti-solvent;
(iii) stirring the inner vial mixture of step (ii) to precipitate solids; and
(iv) filtering the solids of step (iii) and drying the solids under vacuum at room temperature.

In some embodiments, the anti-solvent is pentane.

In some embodiments, the pharmaceutical composition of the invention includes a therapeutically effective amount of the crystalline or solvate form of the invention. In some embodiments, the pharmaceutical composition includes between about 5% (w/w) to about 30% (w/w) of the crystalline or solvate form of the invention. In some embodiments, the pharmaceutical composition includes about 5% (w/w), 10% (w/w), 24% (w/w) or about 25% (w/w) of the crystalline or solvate form of the invention. In some embodiments, the pharmaceutical composition of the invention is a nanosuspension or a spray dried nanosuspension. In some embodiments, the pharmaceutical composition of the invention further includes a pharmaceutically acceptable plasticizer, binder, bulking agent, carrier, excipient, lubricant, disintegrant, and/or surfactant. In some embodiments, the pharmaceutical composition further includes at least one of hypromellose, sodium lauryl sulfate, and lactose monohydrate. In some embodiments, the pharmaceutical composition further includes at least one of croscarmellose and magnesium stearate. In some embodiments, the pharmaceutical composition is in capsule form. In some embodiments, the capsule is selected from a hard hydroxy propyl methylcellulose capsule, hard gelatin capsule, or soft gelatin capsule. In some embodiments, the pharmaceutical composition of the invention includes the components in the following table:

| Component | Weight percentages (w/w) |
|---|---|
| Compound 1 | 5%-30% |
| Binder | 0-10% |
| Surfactant | 0-5% |
| Carrier and bulking agent | 0-95% |
| Disintegrant | 0-10% |
| Lubricant | 0-5% |

provided the amount of binder, surfactant, carrier, disintegrant, and lubricant are not all 0%. In some embodiments, the binder is hypromellose, the surfactant is sodium lauryl sulfate, the carrier and binding agent is lactose monohydrate, the disintegrant is croscarmellose, and the lubricant is magnesium stearate.

In some embodiments, the pharmaceutical composition includes a nanosuspension of Compound 1 (e.g., Form A, Form B, or Form C) having a ×90 of <400 μm, as measured by PSD, e.g., a ×90 of about 300, a ×50 of about 135, and/or a ×10 of 70. In some embodiments, the pharmaceutical composition includes granules comprising a carrier (e.g., lactose) onto which a nanosuspension of Compound 1 (e.g., Form A, Form B, or Form C) is sprayed, e.g., in combination with a bulking agent (e.g., lactose).

In an aspect, the invention features a method for treating Alzheimer's Disease, preventing seizure, reversing synapse loss, or reducing Tau accumulation of a subject in need thereof, including treating the subject with a therapeutically effective amount of Form A, Form B, or Form C of Compound 1. In some embodiments, about 10 mg, 40 mg, 70 mg, 100 mg, 150 mg, or 200 mg of Compound 1 is administered. In some embodiments, Compound 1 is administered orally.

### BRIEF DESCRIPTION OF THE DRAWINGS

The foregoing and other objects, features and advantages will be apparent from the following description of particular embodiments of the disclosure, as illustrated in the accompanying drawings in which like reference characters refer to the same parts throughout the different views. The drawings are not necessarily to scale, emphasis instead being placed upon illustrating the principles of various embodiments of the disclosure.

FIG. 1 shows the dose response of Compound 1 displacement of [¹⁸F]FPEB.

FIG. 2 shows Compound 1 treatment prevents PAM-induced seizures.

FIG. 3 shows XRPD data of Compound 1 AH Form A.

FIG. 4 shows TGA/DSC data of Compound 1 AH Form A. TGA showed a decomposition event that occurred above 250° C. DSC trace showed a single endothermic event with onset at 135.1° C. and peak apex at 136.5° C. due to the melting of the crystalline material.

FIG. 5 shows XRPD data of Compound 1 Form B.

FIG. 6 shows TGA/DSC data of Compound 1 Form B. TGA showed a desolvation event that occurred up to about 200° C. DSC trace showed an endothermic event with a first onset at about 83.4° C. and peak apex at 90.3° C. due to desolvation, and a second onset at 133.8° C. and peak apex at 136.0° C. due to the melting of the crystalline material.

FIG. 7 shows XRPD data of Compound 1 Form C prepared from a long-term slurry using isopropyl alcohol.

FIG. 8 shows TGA/DSC data of Compound 1 Form C prepared from a long-term slurry using isopropyl alcohol. TGA showed a desolvation event that occurred up to about 200° C. DSC trace showed a first endothermic event with onset at 86.1° C. and peak apex at 89.1° C. due to desolvation, and a second endothermic event with onset at 129.1° C. and peak apex at 131.7° C. due to the melting of the crystalline material.

FIG. 9 shows XRPD data of Compound 1 Form C prepared from vapor diffusion using isopropyl alcohol.

FIG. 10 shows TGA/DSC data of Compound 1 Form C prepared from vapor diffusion using isopropyl alcohol. TGA showed a desolvation event that occurred up to about 200° C. DSC trace showed a first endothermic event with onset at 78.7.1° C. and peak apex at 89.4° C., and a second endothermic event with onset at 132.7° C. and peak apex at 134.2° C.

### DETAILED DESCRIPTION

The present disclosure provides compounds and compositions and methods of using the compounds and compositions for modulating the mGluR5 receptor. The mGluR5 receptor is expressed broadly throughout the CNS with predominant post-synaptic localization, although pre-synaptic expression is also present. It is a Gαq-coupled receptor activating phospholipase C and elevating intracellular calcium levels, leading to activation of downstream signaling molecules. Many studies have demonstrated a role for the receptor in regulating N-methyl-D-aspartate (NMDA) receptor activity as well as synaptic plasticity, suggesting this receptor plays a key role in glutamatergic signal transduction.

The compounds of the present disclosure bind to mGluR5 and are allosteric modulators of mGluR5. In some embodiments, the compounds are mGluR5 silent allosteric modulators. Thus, the compounds and compositions described herein can be administered to modulate mGluR5 and to treat individual subjects having a disease, disorder and/or condition related to mGluR5, such as, but not limited to, CNS disorders.

### I. Compounds of the Disclosure

The compound of the present disclosure is (4R,5R)-5-(2-chlorophenyl)-4-(5-(phenylethynyl)pyridin-3-yl)oxazolidin-2-one

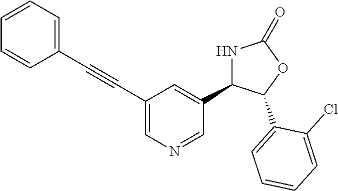 [SMILES: O=C1N[C@H](c2cncc(C#Cc3ccccc3)c2)[C@@H](c2ccccc2Cl)O1]

(Compound 1) or a pharmaceutically acceptable salt thereof, as described in U.S. Pat. No. 8,691,821, hereby incorporated by reference.

The compound of the present disclosure may be prepared using any convenient methodology known to a person of the art, such as the method disclosed in U.S. Pat. No. 8,691,821.

### Solid Forms of Compound 1

Solids exist in either amorphous or crystalline forms. Polymorphism relates to various crystalline forms of a chemical substance. These solid forms and crystalline forms have different characteristics in structures and physical properties, such as XRPD spectrum, IR spectrum, and melting point. A particular solid form or polymorph form may have advantages over other forms and may be more suitable for the manufacture and use of the drug substance.

An anhydrous (AH) form (Form A), two solvated forms (Solvates S1 and S2, interchangeably referred to as Form B and Form C, respectively), and multiple amorphous forms of Compound 1 have been discovered. The amorphous forms resulted in a gel after evaporation in various solvent systems. The anhydrous Form A is the most stable form, e.g., as determined by DSC and TGA analyses.

AH Form A can be identified with single crystal X-ray Powder Diffraction (XRPD) by irradiation with Cu Kα X-rays. The main peaks of Form A were identified, and their relative intensities are listed in Table 1. As will be understood by a person skilled in the art, the relative intensities of the peaks within Table 1 may vary due to various factors such as the purity of the material being analyzed, orientation effects of crystals in the X-ray beam, the degree of crystallinity of the sample, and so on. The peak positions may also shift for variations of sample height, but the peak positions will remain substantially as defined in Table 1. A person skilled in the art will also understand that measurements using a different wavelength will result in different shifts according to the Bragg equation (nλ=2d sin θ). Such further XRPD patterns generated by use of alternative wavelengths are alternative representations of the XRPD patterns of the crystalline materials.

TABLE 1
XRPD peak listing for Form A

| Scan 1 | | | | Scan 2 | | | | | Average |
|---|---|---|---|---|---|---|---|---|---|
| Pos. | Height | d-spacing | Rel. | Pos. | Height | d-spacing | Rel. | AveragePos. | d-spacing |
| [°2θ] | [cts] | [Å] | Int. [%] | [°2θ] | [cts] | [Å] | Int. [%] | [°2θ] | [Å] |
| 6.74 | 2780.79 | 13.11 | 37.19 | 6.77 | 965.33 | 13.05 | 36.02 | 6.75 | 13.08 |
| 6.87 | 1591.51 | 12.86 | 21.28 | 6.89 | 427.17 | 12.82 | 15.94 | 6.88 | 12.84 |
| 9.01 | 2852.25 | 9.81 | 38.15 | 9.03 | 903.15 | 9.79 | 33.70 | 9.02 | 9.80 |
| 11.65 | 2579.35 | 7.59 | 34.50 | 11.66 | 1026.93 | 7.58 | 38.32 | 11.65 | 7.59 |
| 11.85 | 2702.90 | 7.46 | 36.15 | 11.87 | 863.80 | 7.45 | 32.24 | 11.86 | 7.46 |
| 12.14 | 1407.87 | 7.29 | 18.83 | 12.16 | 550.33 | 7.27 | 20.54 | 12.15 | 7.28 |
| 13.53 | 3336.83 | 6.54 | 44.63 | 13.56 | 1311.85 | 6.53 | 48.96 | 13.55 | 6.53 |
| 14.98 | 80.72 | 5.91 | 1.08 | 15.00 | 30.92 | 5.90 | 1.15 | 14.99 | 5.90 |
| 15.58 | 405.33 | 5.68 | 5.42 | 15.61 | 150.97 | 5.67 | 5.63 | 15.59 | 5.68 |
| 17.84 | 5285.83 | 4.97 | 70.69 | 17.25 | 40.08 | 5.14 | 1.50 | 17.55 | 5.05 |
| 18.07 | 563.88 | 4.91 | 7.54 | 17.86 | 2378.92 | 4.96 | 88.78 | 17.97 | 4.93 |
| 18.82 | 1346.22 | 4.71 | 18.00 | 18.08 | 215.72 | 4.90 | 8.05 | 18.45 | 4.81 |
| 19.01 | 1481.62 | 4.66 | 19.81 | 18.84 | 557.62 | 4.71 | 20.81 | 18.93 | 4.69 |
| 19.90 | 6593.53 | 4.46 | 88.18 | 19.02 | 587.76 | 4.66 | 21.93 | 19.46 | 4.56 |
| 20.77 | 7477.32 | 4.27 | 100.00 | 19.92 | 2679.63 | 4.45 | 100.00 | 20.34 | 4.36 |
| 21.40 | 874.05 | 4.15 | 11.69 | 20.78 | 2567.51 | 4.27 | 95.82 | 21.09 | 4.21 |
| 21.56 | 413.19 | 4.12 | 5.53 | 21.41 | 337.05 | 4.15 | 12.58 | 21.49 | 4.13 |
| 22.20 | 2651.80 | 4.00 | 35.46 | 21.56 | 184.99 | 4.12 | 6.90 | 21.88 | 4.06 |
| 22.37 | 606.52 | 3.97 | 8.11 | 22.20 | 1161.04 | 4.00 | 43.33 | 22.29 | 3.99 |
| 23.15 | 5660.54 | 3.84 | 75.70 | 22.35 | 416.94 | 3.97 | 15.56 | 22.75 | 3.91 |
| 24.03 | 2770.47 | 3.70 | 37.05 | 23.16 | 2206.67 | 3.84 | 82.35 | 23.59 | 3.77 |
| 24.43 | 1652.16 | 3.64 | 22.10 | 24.05 | 1055.99 | 3.70 | 39.41 | 24.24 | 3.67 |
| 24.63 | 120.01 | 3.61 | 1.60 | 24.44 | 653.73 | 3.64 | 24.40 | 24.54 | 3.63 |
| 25.54 | 356.43 | 3.48 | 4.77 | 24.63 | 334.35 | 3.61 | 12.48 | 25.08 | 3.55 |
| 25.72 | 213.92 | 3.46 | 2.86 | 25.53 | 191.14 | 3.49 | 7.13 | 25.63 | 3.47 |
| 26.14 | 2867.60 | 3.41 | 38.35 | 26.14 | 902.51 | 3.41 | 33.68 | 26.14 | 3.41 |
| 26.64 | 675.40 | 3.34 | 9.03 | 26.23 | 623.52 | 3.39 | 23.27 | 26.44 | 3.37 |
| 26.96 | 656.82 | 3.31 | 8.78 | 26.67 | 294.78 | 3.34 | 11.00 | 26.81 | 3.32 |
| 27.27 | 544.99 | 3.27 | 7.29 | 26.98 | 265.85 | 3.30 | 9.92 | 27.12 | 3.29 |
| 27.76 | 452.52 | 3.21 | 6.05 | 27.29 | 252.78 | 3.27 | 9.43 | 27.53 | 3.24 |
| 28.16 | 1939.19 | 3.17 | 25.93 | 27.78 | 210.61 | 3.21 | 7.86 | 27.97 | 3.19 |
| 28.66 | 638.67 | 3.11 | 8.54 | 28.17 | 819.35 | 3.17 | 30.58 | 28.41 | 3.14 |
| 29.28 | 608.80 | 3.05 | 8.14 | 28.69 | 252.58 | 3.11 | 9.43 | 28.99 | 3.08 |
| 29.60 | 151.87 | 3.02 | 2.03 | 29.30 | 253.45 | 3.05 | 9.46 | 29.45 | 3.03 |
| 30.06 | 327.79 | 2.97 | 4.38 | 29.60 | 68.53 | 3.02 | 2.56 | 29.83 | 2.99 |
| 30.47 | 1132.52 | 2.93 | 15.15 | 30.09 | 103.24 | 2.97 | 3.85 | 30.28 | 2.95 |
| 30.76 | 419.02 | 2.90 | 5.60 | 30.48 | 457.90 | 2.93 | 17.09 | 30.62 | 2.92 |
| 30.94 | 965.50 | 2.89 | 12.91 | 30.80 | 185.44 | 2.90 | 6.92 | 30.87 | 2.89 |
| 31.36 | 307.89 | 2.85 | 4.12 | 30.95 | 444.54 | 2.89 | 16.59 | 31.15 | 2.87 |
| 31.93 | 246.24 | 2.80 | 3.29 | 31.38 | 150.99 | 2.85 | 5.63 | 31.65 | 2.82 |
| 32.58 | 84.71 | 2.75 | 1.13 | 31.93 | 118.39 | 2.80 | 4.42 | 32.25 | 2.77 |
| 33.04 | 260.30 | 2.71 | 3.48 | 32.61 | 56.84 | 2.74 | 2.12 | 32.82 | 2.73 |
| 33.24 | 463.75 | 2.69 | 6.20 | 33.02 | 114.28 | 2.71 | 4.26 | 33.13 | 2.70 |
| 33.31 | 262.78 | 2.69 | 3.51 | 33.28 | 259.31 | 2.69 | 9.68 | 33.29 | 2.69 |
| 34.24 | 233.43 | 2.62 | 3.12 | 34.22 | 96.21 | 2.62 | 3.59 | 34.23 | 2.62 |
| 34.91 | 261.55 | 2.57 | 3.50 | 34.94 | 124.15 | 2.57 | 4.63 | 34.93 | 2.57 |
| 35.76 | 468.82 | 2.51 | 6.27 | 35.77 | 220.59 | 2.51 | 8.23 | 35.76 | 2.51 |
| 36.66 | 405.30 | 2.45 | 5.42 | 36.65 | 191.58 | 2.45 | 7.15 | 36.66 | 2.45 |
| 37.85 | 88.21 | 2.37 | 1.18 | 37.86 | 36.47 | 2.37 | 1.36 | 37.86 | 2.37 |
| 38.65 | 211.71 | 2.33 | 2.83 | 38.62 | 89.21 | 2.33 | 3.33 | 38.64 | 2.33 |
| 39.01 | 146.75 | 2.31 | 1.96 | 39.03 | 52.43 | 2.31 | 1.96 | 39.02 | 2.31 |
| 40.40 | 293.92 | 2.23 | 3.93 | 40.45 | 108.07 | 2.23 | 4.03 | 40.43 | 2.23 |
| 40.98 | 253.43 | 2.20 | 3.39 | 40.99 | 94.52 | 2.20 | 3.53 | 40.99 | 2.20 |
| 41.67 | 190.55 | 2.17 | 2.55 | 41.69 | 89.05 | 2.16 | 3.32 | 41.68 | 2.17 |
| 42.31 | 225.89 | 2.13 | 3.02 | 42.33 | 83.60 | 2.13 | 3.12 | 42.32 | 2.13 |
| 44.01 | 136.98 | 2.06 | 1.83 | 44.06 | 100.21 | 2.05 | 3.74 | 44.03 | 2.05 |

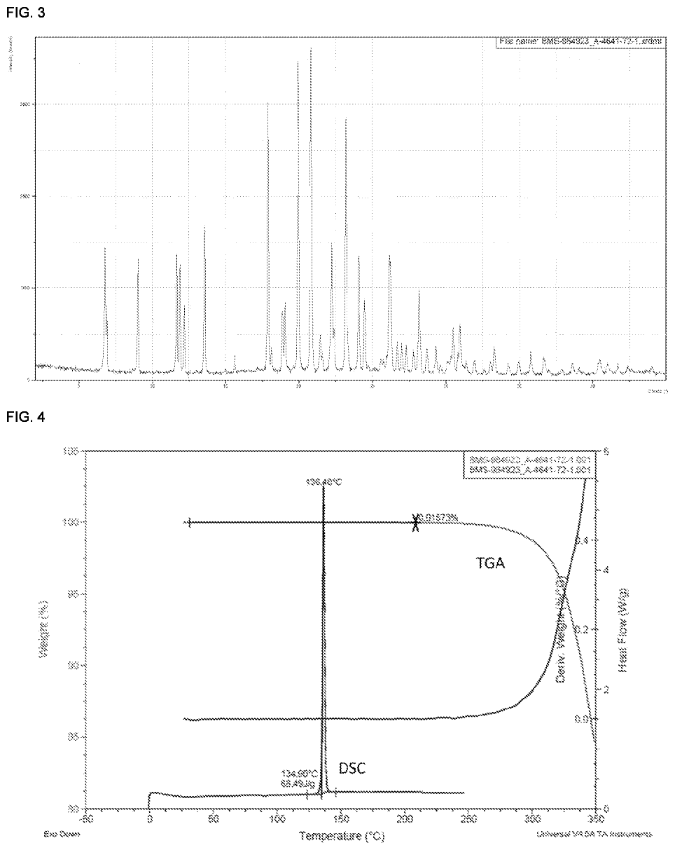

Form A may be characterized by any of its peaks in Table 1. For example, Form A may be characterized by any of the following peaks, among others: 6.75, 6.88, 9.02, 11.65, 11.86, 12.15, 13.55, 14.99, 15.59, 17.55, 17.97, 18.45, 18.93, 19.46, 20.34, 21.09, 21.49, 21.88, 22.29, 22.75, 23.59, 24.24, 24.54, 25.08, 25.63, 26.14, 26.44, 26.81, 27.12, 27.53, 27.97, 28.41, 28.99, 29.45, 29.83, 30.28, 30.62, 30.87, 31.15, 31.65, 32.25, 32.82, 33.13, 33.29, 34.23, 34.93, 35.76, 36.66, 37.86, 38.64, 39.02, 40.43, 40.99, 41.68, 42.32, or 44.03 degrees 2θ, each ±0.2 degrees 2θ. Form A may also be characterized by any of the following d-spacing, among others: 13.08, 12.84, 9.80, 7.59, 7.46, 7.28, 6.53, 5.90, 5.68, 5.05, 4.93, 4.81, 4.69, 4.56, 4.36, 4.21, 4.13, 4.06, 3.99, 3.91, 3.77, 3.67, 3.63, 3.55, 3.47, 3.41, 3.37, 3.32, 3.29, 3.24, 3.19, 3.14, 3.08, 3.03, 2.99, 2.95, 2.92, 2.89, 2.87, 2.82, 2.77, 2.73, 2.70, 2.69, 2.62, 2.57, 2.51, 2.45, 2.37, 2.33, 2.31, 2.23, 2.20, 2.17, 2.13, 2.05 Å, each ±0.2 Å. Form A may also be charactized by an XRPD pattern substantially as shown in FIG. 3.

In some embodiments, the anhydrous crystalline form of Compound 1 is Form A and has an X-ray powder diffraction (XRPD) pattern including one or more peaks of any of 6.75, 6.88, 9.02, 11.65, 11.86, 12.15, 13.55, 14.99, 15.59, 17.55, 17.97, 18.45, 18.93, 19.46, 20.34, 21.09, 21.49, 21.88, 22.29, 22.75, 23.59, 24.24, 24.54, 25.08, 25.63, 26.14, 26.44, 26.81, 27.12, 27.53, 27.97, 28.41, 28.99, 29.45, 29.83, 30.28, 30.62, 30.87, 31.15, 31.65, 32.25, 32.82, 33.13, 33.29, 34.23, 34.93, 35.76, 36.66, 37.86, 38.64, 39.02, 40.43, 40.99, 41.68, 42.32, or 44.03 degrees 2θ, each ±0.2 degrees 2θ.

In some embodiments, the anhydrous crystalline form of Compound 1 is Form A and has an X-ray powder diffraction (XRPD) pattern including all the peaks of 6.75, 6.88, 9.02, 11.65, 11.86, 12.15, 13.55, 14.99, 15.59, 17.55, 17.97, 18.45, 18.93, 19.46, 20.34, 21.09, 21.49, 21.88, 22.29, 22.75, 23.59, 24.24, 24.54, 25.08, 25.63, 26.14, 26.44, 26.81, 27.12, 27.53, 27.97, 28.41, 28.99, 29.45, 29.83, 30.28, 30.62, 30.87, 31.15, 31.65, 32.25, 32.82, 33.13, 33.29, 34.23, 34.93, 35.76, 36.66, 37.86, 38.64, 39.02, 40.43, 40.99, 41.68, 42.32, and 44.03 degrees 2θ, each ±0.2 degrees 2θ.

In some embodiments, the anhydrous crystalline form of Compound 1 is Form A and has an X-ray powder diffraction (XRPD) pattern including the peaks 9.02, 11.65, and 11.86 degrees 2θ, each ±0.2 degrees 2θ. In some embodiments, the anhydrous crystalline form of Compound 1 is Form A and has an X-ray powder diffraction (XRPD) pattern including the peaks 12.15, 14.99, 28.99, and 44.03 degrees 2θ, each ±0.2 degrees 2θ. In some embodiments, the anhydrous crystalline form of Compound 1 is Form A and has an X-ray powder diffraction (XRPD) pattern including three peaks of 2θ angles at 21.09, 21.49, and 21.88 degrees 2θ, each ±0.2 degrees 2θ. In some embodiments, the anhydrous crystalline form of Compound 1 is Form A and has an X-ray powder diffraction (XRPD) pattern including the peaks 9.02, 11.65, 11.86, 12.15, 14.99, 21.09, 21.49, 21.88, 28.99, and 44.03 degrees 2θ, each ±0.2 degrees 2θ.

In some embodiments, Form A analysed by differential scanning calorimetry (DSC) thermogram shows a melting endothermic peak at about 134° C.-138° C. as shown in FIG. 4.

In some embodiments, Form A analysed by thermogravimetric analysis (TGA) exhibits a dehydration from ambient to about 250° C. with a weight loss of about 0.02% (w/w) as shown in FIG. 4.

Some embodiments provide a method for preparing an anhydrous (AH) crystalline form of Compound 1, including precipitating the anhydrous crystalline form from a solution including Compound 1 and a solvent selected from the group consisting of ethyl acetate (EtOAc), heptane, and mixtures thereof. In some embodiments, the solvent may be a mixture of EtOAc and heptane. The ratio of EtOAc and heptane is 90:10, 80:20, 70:30, 60:40, 50:50, 40:60, 30:70, 20:80 or 10:90. The method may further include cooling the solution and/or isolating the AH crystalline form by filtration.

Form B can be identified with single crystal X-ray Powder Diffraction (XRPD) by irradiation with Cu Kα X-rays. The main peaks of Form B were identified, and their relative intensities are listed in Table 2. As will be understood by a person skilled in the art, the relative intensities of the peaks within Table 2 may vary due to various factors such as the purity of the material being analyzed, orientation effects of crystals in the X-ray beam, the degree of crystallinity of the sample, and so on. The peak positions may also shift for variations of sample height, but the peak positions will remain substantially as defined in Table 2. A person skilled in the art will also understand that measurements using a different wavelength will result in different shifts according to the Bragg equation (nλ=2d sin θ). Such further XRPD patterns generated by use of alternative wavelengths are alternative representations of the XRPD patterns of the crystalline materials.

TABLE 2
XRPD peak listing for Form B

| Pos. [°2θ] | Height [cts] | d-spacing [Å] | Rel. Int. [%] |
|---|---|---|---|
| 3.36 | 61.60 | 26.31 | 1.75 |
| 6.75 | 1416.59 | 13.09 | 40.26 |
| 6.85 | 509.62 | 12.90 | 14.48 |
| 8.19 | 369.60 | 10.79 | 10.5 |
| 10.14 | 11.35 | 8.72 | 0.32 |
| 10.84 | 253.71 | 8.15 | 7.21 |
| 13.18 | 122.51 | 6.71 | 3.48 |
| 13.40 | 117.88 | 6.60 | 3.35 |
| 13.54 | 282.19 | 6.54 | 8.02 |
| 13.70 | 1567.79 | 6.46 | 44.56 |
| 15.65 | 50.32 | 5.66 | 1.43 |
| 16.07 | 429.49 | 5.51 | 12.21 |
| 16.27 | 3518.31 | 5.45 | 100 |
| 16.58 | 1033.66 | 5.34 | 29.38 |
| 16.77 | 122.09 | 5.28 | 3.47 |
| 17.15 | 87.86 | 5.17 | 2.5 |
| 17.74 | 126.15 | 4.99 | 3.59 |
| 18.63 | 659.62 | 4.76 | 18.75 |
| 18.73 | 310.84 | 4.73 | 8.83 |
| 19.16 | 573.48 | 4.63 | 16.3 |
| 19.44 | 1293.36 | 4.56 | 36.76 |
| 19.63 | 857.16 | 4.52 | 24.36 |
| 19.95 | 432.1 | 4.45 | 12.28 |
| 20.21 | 349.07 | 4.39 | 9.92 |
| 20.36 | 354.79 | 4.36 | 10.08 |
| 20.60 | 175.19 | 4.31 | 4.98 |
| 21.56 | 1694.51 | 4.12 | 48.16 |
| 21.78 | 1489.37 | 4.08 | 42.33 |
| 22.42 | 648.49 | 3.96 | 18.43 |
| 22.88 | 924.23 | 3.88 | 26.27 |
| 23.06 | 506.07 | 3.85 | 14.38 |
| 23.22 | 1210.94 | 3.83 | 34.42 |
| 23.43 | 249.07 | 3.79 | 7.08 |
| 23.75 | 627.13 | 3.74 | 17.82 |
| 23.99 | 2134.33 | 3.71 | 60.66 |
| 24.53 | 910.49 | 3.63 | 25.88 |
| 24.88 | 426.02 | 3.58 | 12.11 |
| 25.23 | 249.95 | 3.53 | 7.1 |
| 25.43 | 559.47 | 3.50 | 15.9 |
| 26.03 | 482.33 | 3.42 | 13.71 |
| 26.63 | 511.02 | 3.34 | 14.52 |
| 26.89 | 560.15 | 3.31 | 15.92 |
| 27.27 | 38.44 | 3.27 | 1.09 |
| 27.70 | 28.66 | 3.22 | 0.81 |
| 28.22 | 681.80 | 3.16 | 19.38 |
| 29.44 | 97.09 | 3.03 | 2.76 |
| 29.95 | 56.56 | 2.98 | 1.61 |
| 30.24 | 37.57 | 2.95 | 1.07 |
| 30.51 | 82.92 | 2.93 | 2.36 |
| 30.71 | 66.32 | 2.91 | 1.89 |
| 31.14 | 283.87 | 2.87 | 8.07 |
| 31.72 | 164.07 | 2.82 | 4.66 |
| 32.01 | 98.64 | 2.79 | 2.8 |
| 32.27 | 242.08 | 2.77 | 6.88 |
| 32.47 | 448.45 | 2.75 | 12.75 |
| 32.85 | 170.23 | 2.72 | 4.84 |
| 33.48 | 65.35 | 2.67 | 1.86 |
| 33.62 | 129.03 | 2.66 | 3.67 |
| 34.15 | 204.29 | 2.62 | 5.81 |
| 34.63 | 278.19 | 2.59 | 7.91 |
| 35.23 | 48.56 | 2.55 | 1.38 |
| 35.91 | 65.65 | 2.50 | 1.87 |
| 36.16 | 89.48 | 2.48 | 2.54 |
| 36.71 | 81.75 | 2.45 | 2.32 |
| 37.31 | 75.67 | 2.41 | 2.15 |
| 37.79 | 61.02 | 2.38 | 1.73 |
| 38.20 | 119.43 | 2.35 | 3.39 |
| 38.83 | 38.49 | 2.32 | 1.09 |
| 39.92 | 44.75 | 2.26 | 1.27 |
| 40.26 | 95 | 2.24 | 2.7 |
| 40.58 | 74.17 | 2.22 | 2.11 |
| 41.37 | 31.17 | 2.18 | 0.89 |
| 42.56 | 74.2 | 2.12 | 2.11 |
| 43.38 | 50.74 | 2.08 | 1.44 |
| 44.67 | 26.4 | 2.03 | 0.75 |

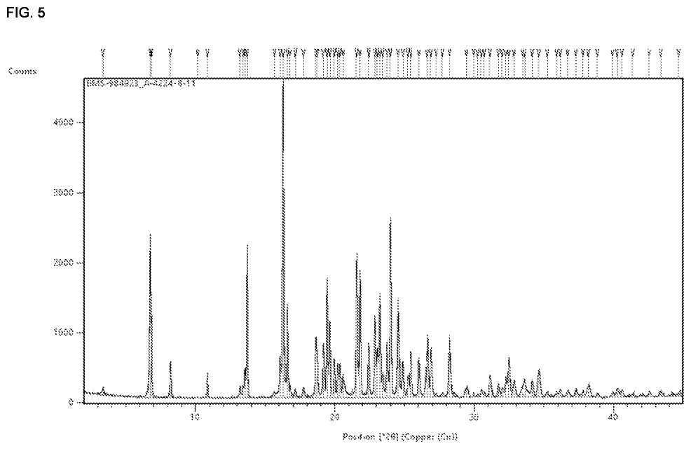

Form B may be characterized by any of its peaks in Table 2. For example, Form B may be characterized by any of the following peaks, among others: 3.36, 6.75, 6.85, 8.19, 10.14, 10.84, 13.18, 13.4, 13.54, 13.7, 15.65, 16.07, 16.27, 16.58, 16.77, 17.15, 17.74, 18.63, 18.73, 19.16, 19.44, 19.63, 19.95, 20.21, 20.36, 20.6, 21.56, 21.78, 22.42, 22.88, 23.06, 23.22, 23.43, 23.75, 23.99, 24.53, 24.88, 25.23, 25.43, 26.03, 26.63, 26.89, 27.27, 27.7, 28.22, 29.44, 29.95, 30.24, 30.51, 30.71, 31.14, 31.72, 32.01, 32.27, 32.47, 32.85, 33.48, 33.62, 34.15, 34.63, 35.23, 35.91, 36.16, 36.71, 37.31, 37.79, 38.2, 38.83, 39.92, 40.26, 40.58, 41.37, 42.56, 43.38, or 44.67 degrees 2θ, each ±0.2 degrees 2θ. Form B may also be characterized by any of the following d-spacing, among others: 26.31, 13.09, 12.9, 10.79, 8.72, 8.15, 6.71, 6.6, 6.54, 6.46, 5.66, 5.51, 5.45, 5.34, 5.28, 5.17, 4.99, 4.76, 4.73, 4.63, 4.56, 4.52, 4.45, 4.39, 4.36, 4.31, 4.12, 4.08, 3.96, 3.88, 3.85, 3.83, 3.79, 3.74, 3.71, 3.63, 3.58, 3.53, 3.5, 3.42, 3.34, 3.31, 3.27, 3.22, 3.16, 3.03, 2.98, 2.95, 2.93, 2.91, 2.87, 2.82, 2.79, 2.77, 2.75, 2.72, 2.67, 2.66, 2.62, 2.59, 2.55, 2.5, 2.48, 2.45, 2.41, 2.38, 2.35, 2.32, 2.26, 2.24, 2.22, 2.18, 2.12, 2.08, or 2.03, each ±0.2 Å. Form B may also be charactized by an XRPD pattern substantially as shown in FIG. 5.

In some embodiments, the solvate form of Compound 1 is Form B and has an X-ray powder diffraction (XRPD) pattern including one or more peaks of any of 3.36, 6.75, 6.85, 8.19, 10.14, 10.84, 13.18, 13.4, 13.54, 13.7, 15.65, 16.07, 16.27, 16.58, 16.77, 17.15, 17.74, 18.63, 18.73, 19.16, 19.44, 19.63, 19.95, 20.21, 20.36, 20.6, 21.56, 21.78, 22.42, 22.88, 23.06, 23.22, 23.43, 23.75, 23.99, 24.53, 24.88, 25.23, 25.43, 26.03, 26.63, 26.89, 27.27, 27.7, 28.22, 29.44, 29.95, 30.24, 30.51, 30.71, 31.14, 31.72, 32.01, 32.27, 32.47, 32.85, 33.48, 33.62, 34.15, 34.63, 35.23, 35.91, 36.16, 36.71, 37.31, 37.79, 38.2, 38.83, 39.92, 40.26, 40.58, 41.37, 42.56, 43.38, or 44.67 degrees 2θ, each ±0.2 degrees 2θ.

In some embodiments, the solvate form of Compound 1 is Form B and has an X-ray powder diffraction (XRPD) pattern including all the peaks of 3.36, 6.75, 6.85, 8.19, 10.14, 10.84, 13.18, 13.4, 13.54, 13.7, 15.65, 16.07, 16.27, 16.58, 16.77, 17.15, 17.74, 18.63, 18.73, 19.16, 19.44, 19.63, 19.95, 20.21, 20.36, 20.6, 21.56, 21.78, 22.42, 22.88, 23.06, 23.22, 23.43, 23.75, 23.99, 24.53, 24.88, 25.23, 25.43, 26.03, 26.63, 26.89, 27.27, 27.7, 28.22, 29.44, 29.95, 30.24, 30.51, 30.71, 31.14, 31.72, 32.01, 32.27, 32.47, 32.85, 33.48, 33.62, 34.15, 34.63, 35.23, 35.91, 36.16, 36.71, 37.31, 37.79, 38.2, 38.83, 39.92, 40.26, 40.58, 41.37, 42.56, 43.38, and 44.67 degrees 2θ, each ±0.2 degrees 2θ.

In some embodiments, the solvate form of Compound 1 is Form B and has an X-ray powder diffraction (XRPD) pattern including at least three of the following peaks of 2θ angles at 16.07, 16.27, 16.58, and 16.77 degrees 2θ, each ±0.2 degrees 2θ. In some embodiments, the solvate form of Compound 1 is Form B and has an X-ray powder diffraction (XRPD) pattern including three or more of the following peaks of 2θ angles at 13.18, 13.4, 13.54, 13.54, and 13.70 degrees 2θ, each ±0.2 degrees 2θ. In some embodiments, the solvate form of Compound 1 is Form B and has an X-ray powder diffraction (XRPD) pattern including peaks at 37.31, and 39.92 degrees 2θ, each ±0.2 degrees 2θ. In some embodiments, the solvate form of Compound 1 is Form B and has an X-ray powder diffraction (XRPD) pattern including the following peaks of 2θ angles at 13.18, 13.4, 13.54, 13.54, 13.70, 16.07, 16.27, 16.58, 16.77, 37.31, and 39.92 degrees 2θ, each ±0.2 degrees 2θ. In some embodiments, the solvate form of Compound 1 is Form B and has an X-ray powder diffraction (XRPD) pattern including one, two, three, four, five, six, seven, eight, or all of the following peaks of 2θ angles at 15.65, 17.74, 27.7, 31.14, 37.79, 38.83, 40.23, 40.58, 41.37 degrees 2θ, each ±0.2 degrees 2θ. In some embodiments, the solvate form of Compound 1 is Form B and has an X-ray powder diffraction (XRPD) pattern including one, two, three, four, or all of the following peaks of 2θ angles at 3.36, 8.19, 10.84, 43.38, and 44.67 degrees 2θ, each ±0.2 degrees 2θ.

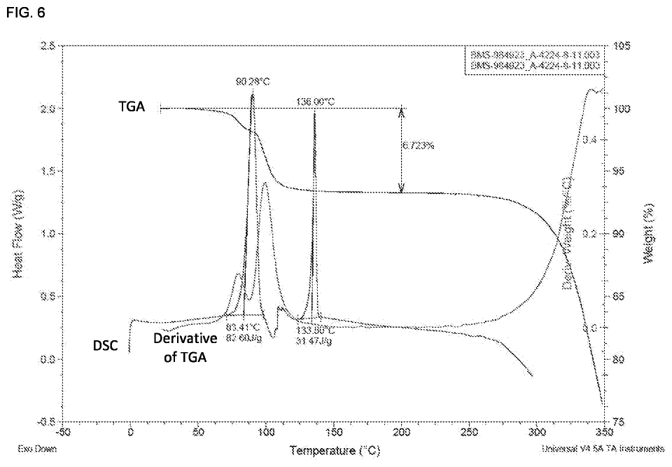

In some embodiments, Form B analysed by differential scanning calorimetry (DSC) thermogram shows a melting endothermic peak at about 134-138° C. as shown in FIG. 6.

In some embodiments, Form B analysed by thermogravimetric analysis (TGA) exhibits a desolvation from ambient to about 200° C. with a weight loss of about 6.7% (w/w) as shown in FIG. 6. Some embodiments provide a method for preparing Form B of Compound 1, including the evaporation of a mixture of Compound 1 and isopropyl alcohol under inert atmosphere, e.g., nitrogen gas, in a dry-box. In some embodiments, the evaporation may occur over a period of 1-2 weeks. In some embodiments, the method may further include drying the crystalline Form B under vacuum.

Form C, prepared from a long-term slurry using isopropyl alcohol, can be identified with single crystal XRPD by irradiation with Cu Kα X-rays. The main peaks of Form C were identified, and their relative intensities are listed in Table 3. As will be understood by a person skilled in the art, the relative intensities of the peaks within Tables 3 may vary due to various factors such as the purity of the material being analyzed, orientation effects of crystals in the X-ray beam, the degree of crystallinity of the sample, and so on. The peak positions may also shift for variations of sample height, but the peak positions will remain substantially as defined in Table 3. A person skilled in the art will also understand that measurements using a different wavelength will result in different shifts according to the Bragg equation (nλ=2d sin θ). Such further XRPD patterns generated by use of alternative wavelengths are alternative representations of the XRPD patterns of the crystalline materials.

TABLE 3
XRPD peak listing for Form C prepared from
a long-term slurry using isopropyl alcohol

| Pos. [°2θ] | Height [cts] | d-spacing [Å] | Rel. Int. [%] |
|---|---|---|---|
| 3.40 | 114.39 | 25.93 | 5.51 |
| 6.83 | 2075.69 | 12.92 | 100.00 |
| 8.33 | 330.50 | 10.61 | 15.92 |
| 10.94 | 95.09 | 8.08 | 4.58 |
| 13.32 | 143.33 | 6.64 | 6.90 |
| 13.82 | 597.53 | 6.40 | 28.79 |
| 16.32 | 1503.18 | 5.43 | 72.42 |
| 16.66 | 591.16 | 5.32 | 28.48 |
| 16.95 | 121.30 | 5.23 | 5.84 |
| 17.24 | 69.38 | 5.14 | 3.34 |
| 18.76 | 448.85 | 4.73 | 21.62 |
| 19.27 | 208.51 | 4.60 | 10.05 |
| 19.60 | 660.49 | 4.53 | 31.82 |
| 20.13 | 453.96 | 4.41 | 21.87 |
| 20.31 | 282.83 | 4.37 | 13.63 |
| 20.81 | 195.56 | 4.26 | 9.42 |
| 21.86 | 764.5 | 4.06 | 36.83 |
| 22.72 | 172.54 | 3.91 | 8.31 |
| 23.15 | 728.32 | 3.84 | 35.09 |
| 23.38 | 78.88 | 3.80 | 3.80 |
| 23.57 | 327.26 | 3.77 | 15.77 |
| 23.86 | 405.15 | 3.73 | 19.52 |
| 24.30 | 328.60 | 3.66 | 15.83 |
| 24.89 | 330.49 | 3.57 | 15.92 |
| 25.61 | 299.01 | 3.48 | 14.41 |
| 26.24 | 128.37 | 3.39 | 6.18 |
| 26.92 | 139.98 | 3.31 | 6.74 |
| 27.28 | 132.49 | 3.27 | 6.38 |
| 28.53 | 172.30 | 3.13 | 8.30 |
| 29.51 | 107.49 | 3.02 | 5.18 |
| 30.63 | 56.99 | 2.92 | 2.75 |
| 31.64 | 50.31 | 2.83 | 2.42 |
| 32.22 | 41.39 | 2.78 | 1.99 |
| 32.59 | 207.50 | 2.75 | 10.00 |
| 32.96 | 105.52 | 2.72 | 5.08 |
| 33.72 | 107.37 | 2.66 | 5.17 |
| 34.34 | 74.88 | 2.61 | 3.61 |
| 34.80 | 162.69 | 2.58 | 7.84 |
| 35.36 | 34.06 | 2.54 | 1.64 |
| 36.25 | 52.56 | 2.48 | 2.53 |
| 36.91 | 66.84 | 2.43 | 3.22 |
| 38.24 | 37.92 | 2.35 | 1.83 |
| 42.75 | 37.28 | 2.11 | 1.80 |
| 43.52 | 17.75 | 2.08 | 0.85 |
| 44.99 | 11.58 | 2.01 | 0.56 |

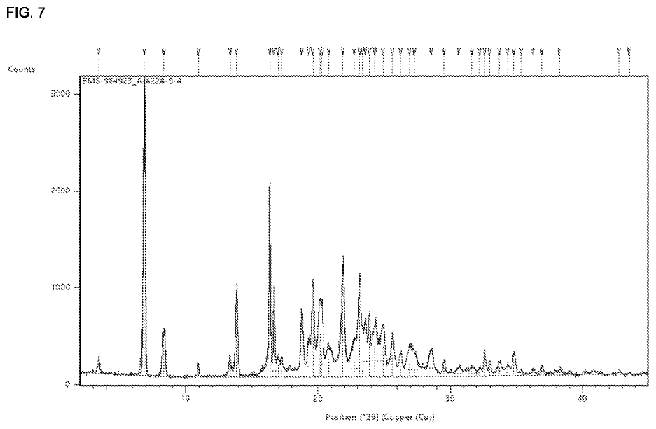

Form C, prepared from a long-term slurry using isopropyl alcohol, may be characterized by any of its peaks in Table 3. For example, Form C may be characterized by any of the following peaks, among others: 3.40, 6.83, 8.33, 10.94, 13.32, 13.82, 16.32, 16.66, 16.95, 17.24, 18.76, 19.27, 19.6, 20.13, 20.31, 20.81, 21.86, 22.72, 23.15, 23.38, 23.57, 23.86, 24.30, 24.89, 25.61, 26.24, 26.92, 27.28, 28.53, 29.51, 30.63, 31.64, 32.22, 32.59, 32.96, 33.72, 34.34, 34.8, 35.36, 36.25, 36.91, 38.24, 42.75, 43.52, or 44.99 degrees 2θ, each ±0.2 degrees 2θ. Form C, prepared from a long-term slurry from isopropyl alcohol, may also be characterized by any of the following d-spacing, among others: 25.93, 12.92, 10.61, 8.08, 6.64, 6.4, 5.43, 5.32, 5.23, 5.14, 4.73, 4.6, 4.53, 4.41, 4.37, 4.26, 4.06, 3.91, 3.84, 3.8, 3.77, 3.73, 3.66, 3.57, 3.48, 3.39, 3.31, 3.27, 3.13, 3.02, 2.92, 2.83, 2.78, 2.75, 2.72, 2.66, 2.61, 2.58, 2.54, 2.48, 2.43, 2.35, 2.11, 2.08, or 2.01, each ±0.2 Å. Form C, prepared from a long-term slurry using isopropyl alcohol, may also be charactized by an XRPD pattern substantially as shown in FIG. 7.

In some embodiments, the solvate form of Compound 1 is Form C, prepared from a long-term slurry using isopropyl alcohol, and has an X-ray powder diffraction (XRPD) pattern including one or more peaks of any of 3.40, 6.83, 8.33, 10.94, 13.32, 13.82, 16.32, 16.66, 16.95, 17.24, 18.76, 19.27, 19.6, 20.13, 20.31, 20.81, 21.86, 22.72, 23.15, 23.38, 23.57, 23.86, 24.30, 24.89, 25.61, 26.24, 26.92, 27.28, 28.53, 29.51, 30.63, 31.64, 32.22, 32.59, 32.96, 33.72, 34.34, 34.8, 35.36, 36.25, 36.91, 38.24, 42.75, 43.52, or 44.99 degrees 2θ, each ±0.2 degrees 2θ.

In some embodiments, the solvate form of Compound 1 is Form C, prepared from a long-term slurry using isopropyl alcohol, and has an X-ray powder diffraction (XRPD) pattern including all the peaks of 3.40, 6.83, 8.33, 10.94, 13.32, 13.82, 16.32, 16.66, 16.95, 17.24, 18.76, 19.27, 19.6, 20.13, 20.31, 20.81, 21.86, 22.72, 23.15, 23.38, 23.57, 23.86, 24.30, 24.89, 25.61, 26.24, 26.92, 27.28, 28.53, 29.51, 30.63, 31.64, 32.22, 32.59, 32.96, 33.72, 34.34, 34.8, 35.36, 36.25, 36.91, 38.24, 42.75, 43.52, and 44.99 degrees 2θ, each ±0.2 degrees 2θ.

In some embodiments, the solvate form of Compound 1 is Form C, prepared from a long-term slurry using isopropyl alcohol, and has an X-ray powder diffraction (XRPD) pattern including only two peaks between 13.32 and 13.82 degrees 2θ, each ±0.2 degrees 2θ, e.g., at 13.32 and 13.82, degrees 2θ, each ±0.2 degrees 2θ. In some embodiments, the solvate form of Compound 1 is Form C, prepared from a long-term slurry using isopropyl alcohol, and has an X-ray powder diffraction (XRPD) pattern including only three peaks between 32.22 and 32.96 degrees 2θ, each ±0.2 degrees 2θ, e.g., at 32.22, 32.59, and 32.96 degrees 2θ, each ±0.2 degrees 2. In some embodiments, the solvate form of Compound 1 is Form C, prepared from a long-term slurry using isopropyl alcohol, and has an X-ray powder diffraction (XRPD) pattern including peaks at 3.4, 8.33, 10.94, 16.32, and 16.66 degrees 2θ, each ±0.2 degrees 2θ. In some embodiments, the solvate form of Compound 1 is Form C, prepared from a long-term slurry using isopropyl alcohol, and has an X-ray powder diffraction (XRPD) pattern including peaks at 3.4, 8.33, 10.94, 13.32, 13.82, 16.32, 16.66, 32.22, 32.59, 32.96 degrees 2θ, each ±0.2 degrees 2θ. In some embodiments, the solvate form of Compound 1 is Form C, prepared from a long-term slurry using isopropyl alcohol, and has an X-ray powder diffraction (XRPD) pattern including peaks at 16.95, 36.25, 42.75, 43.52, and 44.99 degrees 2θ, each ±0.2 degrees 2θ.

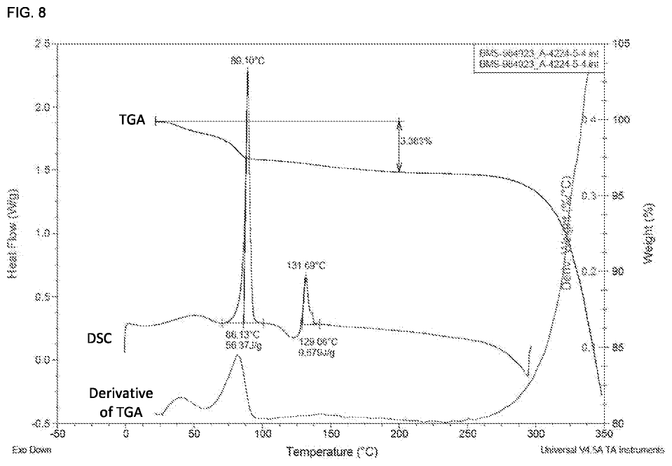

In some embodiments, Form C, prepared from a long-term slurry using isopropyl alcohol, analysed by differential scanning calorimetry (DSC) thermogram shows a melting endothermic peak at about 129-133° C. as shown in FIG. 8.

In some embodiments, Form C, prepared from a long-term slurry using isopropyl alcohol, analysed by thermogravimetric analysis (TGA) exhibits a dehydration from ambient to about 200° C. with a weight loss of about 3.4% (w/w) as shown in FIG. 8.

Some embodiments provide a process for preparing a Form C of Compound 1, including the precipitation of the solvate form from a solution including Compound 1 and isopropyl alcohol. In some embodiments, the solution may be stirred at 10-30° C., e.g., 20° C., over two weeks. The process may further include isolating the solvate by filtration.

Form C, prepared from vapor diffusion using isopropyl alcohol, can be identified with single crystal XRPD by irradiation with Cu Kα X-rays. The main peaks of Form C were identified, and their relative intensities are listed in Table 4. As will be understood by a person skilled in the art, the relative intensities of the peaks within Table 4 may vary due to various factors such as the purity of the material being analyzed, orientation effects of crystals in the X-ray beam, the degree of crystallinity of the sample, and so on. The peak positions may also shift for variations of sample height, but the peak positions will remain substantially as defined in Table 4. A person skilled in the art will also understand that measurements using a different wavelength will result in different shifts according to the Bragg equation (nλ=2d sin θ). Such further XRPD patterns generated by use of alternative wavelengths are alternative representations of the XRPD patterns of the crystalline materials.

TABLE 4
XRPD peak listing for Form C prepared from
vapor diffusion using isopropyl alcohol

| Pos. [°2θ] | Height [cts] | d-spacing [Å] | Rel. Int. [%] |
|---|---|---|---|
| 3.38 | 28.72 | 26.12 | 2.76 |
| 6.85 | 1041.25 | 12.90 | 100.00 |
| 8.35 | 136.82 | 10.58 | 13.14 |
| 10.95 | 26.05 | 8.07 | 2.50 |
| 13.31 | 71.42 | 6.65 | 6.86 |
| 13.84 | 224.21 | 6.39 | 21.53 |
| 16.31 | 638.67 | 5.43 | 61.34 |
| 16.65 | 260.45 | 5.32 | 25.01 |
| 16.99 | 66.38 | 5.21 | 6.38 |
| 17.23 | 45.42 | 5.14 | 4.36 |
| 18.76 | 176.23 | 4.73 | 16.93 |
| 19.33 | 105.99 | 4.59 | 10.18 |
| 19.60 | 227.91 | 4.53 | 21.89 |
| 20.13 | 258.97 | 4.41 | 24.87 |
| 20.30 | 135.22 | 4.37 | 12.99 |
| 20.80 | 75.82 | 4.27 | 7.28 |
| 21.05 | 30.05 | 4.22 | 2.89 |
| 21.86 | 307.79 | 4.06 | 29.56 |
| 22.68 | 86.26 | 3.92 | 8.28 |
| 23.13 | 237.36 | 3.84 | 22.80 |
| 23.60 | 131.05 | 3.77 | 12.59 |
| 23.88 | 158.79 | 3.72 | 15.25 |
| 24.30 | 158.25 | 3.66 | 15.20 |
| 24.90 | 134.33 | 3.57 | 12.90 |
| 25.64 | 150.40 | 3.47 | 14.44 |
| 26.24 | 59.59 | 3.39 | 5.72 |
| 26.88 | 54.13 | 3.31 | 5.20 |
| 27.26 | 59.17 | 3.27 | 5.68 |
| 28.00 | 31.11 | 3.18 | 2.99 |
| 28.53 | 79.40 | 3.13 | 7.63 |
| 29.53 | 68.23 | 3.02 | 6.55 |
| 30.66 | 29.9 | 2.91 | 2.87 |
| 31.64 | 23.45 | 2.83 | 2.25 |
| 32.59 | 64.00 | 2.75 | 6.15 |
| 32.95 | 33.61 | 2.72 | 3.23 |
| 33.74 | 37.32 | 2.65 | 3.58 |
| 34.78 | 59.85 | 2.58 | 5.75 |
| 36.21 | 28.95 | 2.48 | 2.78 |
| 36.94 | 54.09 | 2.43 | 5.19 |
| 38.24 | 14.02 | 2.35 | 1.35 |
| 40.17 | 13.28 | 2.24 | 1.28 |
| 40.80 | 12.38 | 2.21 | 1.19 |
| 43.55 | 26.21 | 2.08 | 2.52 |

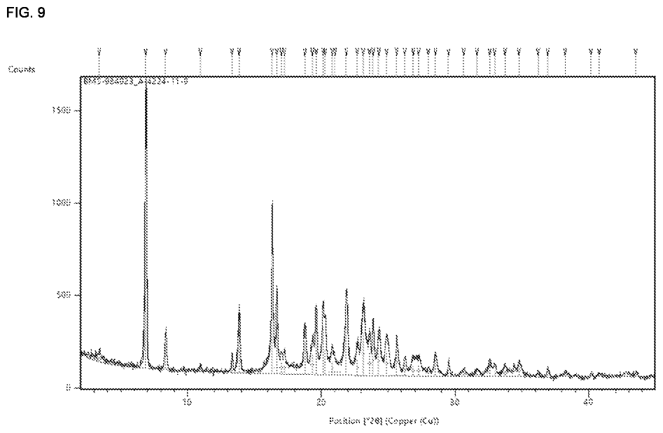

Form C, prepared from vapor diffusion using isopropyl alcohol, may be characterized by any of its peaks in Table 4. For example, Form C may be characterized by any of the following peaks, among others: 3.38, 6.85, 8.35, 10.95, 13.31, 13.84, 16.31, 16.65, 16.99, 17.23, 18.76, 19.33, 19.6, 20.13, 20.30, 20.80, 21.05, 21.86, 22.68, 23.13, 23.6, 23.88, 24.3, 24.9, 25.64, 26.24, 26.88, 27.26, 28, 28.53, 29.53, 30.66, 31.64, 32.59, 32.95, 33.74, 34.78, 36.21, 36.94, 38.24, 40.17, 40.80, or 43.55 degrees 2θ, each ±0.2 degrees 2θ. Form C, prepared from vapor diffusion using isopropyl alcohol, may also be characterized by any of the following d-spacing, among others: 26.12, 12.9, 10.58, 8.07, 6.65, 6.39, 5.43, 5.32, 5.21, 5.14, 4.73, 4.59, 4.53, 4.41, 4.37, 4.27, 4.22, 4.06, 3.92, 3.84, 3.77, 3.72, 3.66, 3.57, 3.47, 3.39, 3.31, 3.27, 3.18, 3.13, 3.02, 2.91, 2.83, 2.75, 2.72, 2.65, 2.58, 2.48, 2.43, 2.35, 2.24, 2.21, or 2.08, each ±0.2 Å. Form C, prepared from vapor diffusion using isopropyl alcohol, may also be charactized by an XRPD pattern substantially as shown in FIG. 9.

In some embodiments, the solvate form of Compound 1 is Form C, prepared from vapor diffusion using isopropyl alcohol, and has an X-ray powder diffraction (XRPD) pattern including one or more peaks of any of 3.38, 6.85, 8.35, 10.95, 13.31, 13.84, 16.31, 16.65, 16.99, 17.23, 18.76, 19.33, 19.6, 20.13, 20.30, 20.80, 21.05, 21.86, 22.68, 23.13, 23.6, 23.88, 24.3, 24.9, 25.64, 26.24, 26.88, 27.26, 28, 28.53, 29.53, 30.66, 31.64, 32.59, 32.95, 33.74, 34.78, 36.21, 36.94, 38.24, 40.17, 40.80, or 43.55 degrees 2θ, each ±0.2 degrees 2θ.

In some embodiments, the solvate form of Compound 1 is Form C, prepared from vapor diffusion using isopropyl alcohol, and has an X-ray powder diffraction (XRPD) pattern including all the peaks of 3.38, 6.85, 8.35, 10.95, 13.31, 13.84, 16.31, 16.65, 16.99, 17.23, 18.76, 19.33, 19.6, 20.13, 20.30, 20.80, 21.05, 21.86, 22.68, 23.13, 23.6, 23.88, 24.3, 24.9, 25.64, 26.24, 26.88, 27.26, 28, 28.53, 29.53, 30.66, 31.64, 32.59, 32.95, 33.74, 34.78, 36.21, 36.94, 38.24, 40.17, 40.80, and 43.55 degrees 2θ, each ±0.2 degrees 2θ.

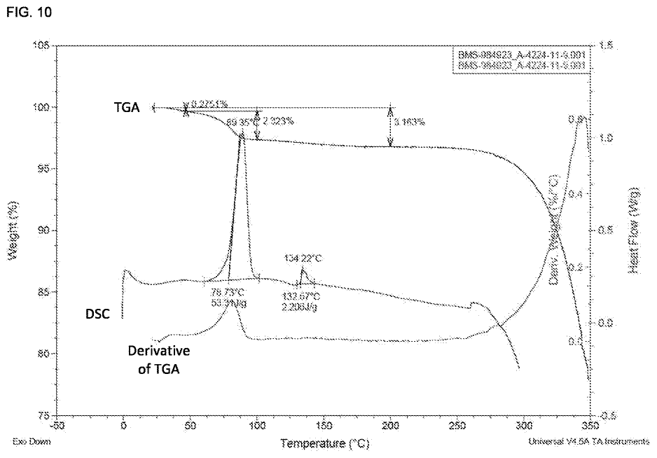

In some embodiments, Form C, prepared from vapor diffusion using isopropyl alcohol, analysed by differential scanning calorimetry (DSC) thermogram shows an endothermic peak at about 89.4° C. and a smaller endothermic peak at about 134.2° C. as shown in FIG. 10.

In some embodiments, Form C, prepared from vapor diffusion using isopropyl alcohol, analysed by thermogravimetric analysis (TGA) exhibits a dehydration at ambient to about 200° C. with a weight loss of about 3.2% (w/w) as shown in FIG. 10.

Some embodiments provide a process for preparing a Form C of Compound 1, including: (i) dissolving Compound 1 in isopropyl alcohol to form a saturated solution; (ii) filtering the mixture of step (i) into a new vial that is placed in an outer vial containing an anti-solvent; (iii) stirring the inner vial mixture of step (ii) to precipitate solids; and (iv) filtering the solids of step (iii) and drying the solids under vacuum at room temperature.

### II. Pharmaceutical Compositions

In some embodiments, compounds and compositions of the present disclosure are administered to humans, human patients or subjects. For the purposes of the present disclosure, the phrase “active ingredient” generally refers to the compounds as described herein. One aspect of the present disclosure provides pharmaceutical compositions including at least one pharmaceutically acceptable carrier and a compound of the present disclosure.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition including a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100%, e.g., between 0.5 and 50%, between 1-30%, between 5-80%, at least 80% (w/w) active ingredient.

The compounds of the present disclosure can be formulated using one or more excipients to: (1) increase stability; (2) permit the sustained or delayed release; (3) alter the biodistribution; (4) alter the release profile of the compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Accordingly, the formulations of the disclosure may include one or more excipients, each in an amount that together increases the stability of the compounds.

### Excipients

Pharmaceutical formulations may include a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly (vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and Kolliphor® (SOLUTOL®)), sucrose fatty acid esters, polyethylene glycol fatty acid esters (e.g. CREMOPHOR®), polyoxyethylene ethers, (e.g. polyoxyethylene lauryl ether [BRIJ®30]), poly (vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER®188, cetrimonium bromide, cetylpyridinium chloride, benzalkonium chloride, docusate sodium, Kolliphor SLS, and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly (vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and combinations thereof.

Exemplary oils include, but are not limited to, almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

In some embodiments, the compositions of the present disclosure include Compound 1, such as the AH crystalline form of Compound 1 (e.g., Form A), a solvate form of Compound 1 (e.g., Form B and Form C), or a mixture thereof, optionally at least one carrier (such as but not limited to lactose monohydrate), optionally at least one disintegrant (such as but not limited to croscarmellose sodium), and/or optionally at least one lubricant (such as but not limited to magnesium stearate). In some embodiments, the composition including Compound 1 is a nanomilled suspension or a spray dried nanosuspension. The composition optionally includes hypromellose (HPMC; such as HPMC 63), sodium lauryl sulfate (SLS; such as Kolliphor SLS), lactose monohydrate, croscarmellose, magnesium stearate, or a mixture thereof. The amount of Compound 1 in these compositions is between about 5% (w/w) to about 30% (w/w), such as about 5% (w/w), about 10% (w/w), about 15% (w/w), about 20% (w/w), about 25% (w/w), or about 30% (w/w). Non-limiting examples of the nano-suspension and active granules compositions are shown in Table 2 below. Non-limiting examples of active capsule compositions are shown in Table 3 below. DL=drug loading.

TABLE 2
Nano-suspension and active granules composition

| | Input nano- | | | | |
|---|---|---|---|---|---|
| | suspension | 25% DL granules | | 5% DL granules | |
| | % (w/w) | g/ batch | % (w/w) | g/ batch | % (w/w) |
| Compound 1 | 10.00 | 250.00 | 25.06 | 70.00 | 5.05 |
| Hypromellose | 2.00 | 50.00 | 5.01 | 14.00 | 1.01 |
| (HPMC 603) | | | | | |
| Sodium lauryl | 0.50 | 12.50 | 1.25 | 3.50 | 0.25 |
| sulfate | | | | | |
| (Kolliphor SLS | | | | | |
| fine) SLS | | | | | |
| Water | 87.50 | Removed during spraying | | | |
| Lactose mono- | | 685.00 | 68.67 | 1300.00 | 93.69 |
| hydrate (11SD) | | | | | |
| TOTAL | 100.00 | 997.50 | 100.00 | 1387.50 | 100.00 |

TABLE 3
Active capsules composition

| | 5 mg strength | | 50 mg strength | | 100 mg strength | |
|---|---|---|---|---|---|---|
| | %(w/w) | mg/cps | %(w/w) | mg/cps | %(w/w) | mg/cps |
| Compound 1 | 4.79 | 5.00 | 23.81 | 50.00 | 23.81 | 100.00 |
| Hypromellose | 0.96 | 1.00 | 4.76 | 10.00 | 4.76 | 20.00 |
| (HPMC 603) | | | | | | |
| Sodium lauryl | 0.24 | 0.25 | 1.19 | 2.50 | 1.19 | 5.00 |
| sulfate | | | | | | |
| (Kolliphor | | | | | | |
| SLS fine) SLS | | | | | | |
| Lactose | 89.01 | 92.86 | 65.24 | 137.00 | 65.24 | 274.00 |
| monohydrate | | | | | | |
| (11SD) | | | | | | |
| Croscarmellose | 4.00 | 4.17 | 4.00 | 8.40 | 4.00 | 16.80 |
| (Ac-di-Sol) | | | | | | |
| Magnesium | 1.00 | 1.04 | 1.00 | 2.10 | 1.00 | 4.20 |
| stearate | | | | | | |
| (Ligamed | | | | | | |
| MF-2-V) | | | | | | |
| TOTAL | 100.00 | 104.32 | 100.00 | 210.00 | 100.00 | 420.00 |

Some embodiments of the present application provide a pharmaceutical composition including an effective amount of Compound 1, such as the AH crystalline form of Compound 1 (e.g., Form A), a solvate form of Compound 1 (e.g., Form B and Form C), or a mixture thereof. In some embodiments, between about 5% (w/w) to about 30% (w/w) of Compound 1 (such as Form A, Form B, or Form C) may be in the pharmaceutical composition. In some embodiments, about 5% (w/w), 10% (w/w), 24% (w/w) or about 25% (w/w) of Compound 1 (such as Form A, Form B, or Form C) may be in the pharmaceutical composition. The pharmaceutical composition may further include at least one of hypromellose (such as HPMC 603), sodium lauryl sulfate (such as Kolliphor SLS), or lactose monohydrate (such as 11SD), and optionally at least one of croscarmellose (such as Ac-di-Sol) or magnesium stearate (such as Ligamed MF-2-V). Examples of weight percentages of the components are shown in Table 4. In some embodiments, the pharmaceutical composition may be in the form of capsules, such as capsules including 5 mg, 50 mg or 100 mg of Compound 1 (e.g., Form A, Form B, or Form C).

TABLE 4
Weight percentages of the components

| Components | Weight percentages |
|---|---|
| Compound 1 (such as | 5%-30% (such as between about 5% to about 10%, between 10% to |
| Form A, Form B, or | about 20%, between about 20% to about 30%; such as about 5%, |
| Form C) | about 10%, about 24%, about 25%) |
| Binder, e.g., | 0-10% (such as between about 1% to about 2%, between about 2% to |
| hypromellose | about 3%, between about 3% to about 4%, between about 4% to about |
| | 5%; such as about 1%, about 2%, about 5%) |
| Surfactant, e.g., | 0-5% (such as between about 0.1% to about 0.5%, between about |
| sodium lauryl | 0.5% to about 1%, between about 1% to about 1.5%; such as about |
| sulfate | 0.2%, 0.25%, about 0.5%, about 1.2%, about 1.25%) |
| Carrier, e.g., | 0-95% (such as between about 50% to about 60%, between about 60% |
| lactose | to about 70%, between about 70% to about 80%, betwnee about 80% |
| monohydrate | to about 90%, between about 90% to about 95%; such as about 65%, |
| | about 68%, about 90%, about 93%) |
| Disintegrant, e.g., | 0-10% (such as between about 1% to about 5%, between about 5% to |
| croscarmellose | about 10%; such as about 4%) |
| Lubricant, e.g., | 0-5% (such as between about 0.5% to about 1.5%; such as about 1%) |
| magnesium stearate | |

### III. Methods of Use

The mGluR5 receptor has emerged as a target of potential therapeutic utility in a number of disease states. Based on the expression pattern and functional role of mGluR5, this receptor is an important target for drug discovery in a number of therapeutic indications. Evaluation of genetically modified mice lacking mGluR5 as well as compounds that modulate receptor function suggest ligands that modulate mGluR5 receptor function have therapeutic utility in CNS and peripheral disease states including, but not limited to, schizophrenia, cognitive impairment, Alzheimer's disease, Parkinson's disease, Parkinson's disease levodopa-induced dyskinesia, addiction, anxiety, depression, psychosis, epilepsy, Fragile X, gastroesophageal reflux disease, migraine, pain, infectious or genetically acquired prion disease (such as but limited to Creutzfeldt-Jakob disease (CJD), Gerstmann-Straussler-Scheinker syndrome (GSS) and fatal familial insomnia), tauopathies (such as but not limited to frontotemporal dementia, corticobasal syndrome, Richardson syndrome, parkinsonism, pure akinesia with gait freezing and, rarely, motor neuron symptoms or cerebellar ataxia), rare neurodegenerative diseases similar to Parkinson's disease, and others.

In some embodiments, a therapeutically effective amount of a compound or composition of the present disclosure can be used for the treatment of CNS disorders and neurological or psychiatric disorders, such as but not limited to, schizophrenia, cognitive impairment, Alzheimer's disease, Parkinson's disease, Parkinson's disease levodopa-induced dyskinesia, addiction, anxiety, depression, psychosis, epilepsy, Fragile X, gastroesophageal reflux disease, migraine, pain, borderline personality disorder, bipolar disorder, or other neurological and/or psychiatric disorder associated with glutamate dysfunction.

In one embodiment, a method for the treatment of Alzheimer's disease is provided, including administering to a patient in need thereof a therapeutically effective amount of a compound or composition of the present disclosure. The compounds may be Compound 1. In some embodiments, a therapeutically effective amount of a compound or composition of the present disclosure is used to reverse synapse density decreases or synapse loss, such as cortical and hippocampal synapse density deceases, of a subject in need thereof. In some embodiments, a therapeutically effective amount of a compound or composition of the present disclosure is used to reduce Tau accumulation of a subject in need thereof.

In some embodiments, a method of preventing seizure is provided, including administering to a patient in need thereof a therapeutically effective amount of a compound or composition of the present disclosure. The compounds may be Compound 1.

In some embodiments, the compounds and compositions of the present disclosure are used in the manufacture of a medicament for the treatment of CNS disorders and neurological or psychiatric disorders, such as but not limited to schizophrenia, cognitive impairment, Alzheimer's disease, Parkinson's disease, Parkinson's disease levodopa-induced dyskinesia, addiction, anxiety, depression, psychosis, epilepsy, Fragile X, gastroesophageal reflux disease, migraine, pain, borderline personality disorder, bipolar disorder, or other neurological and/or psychiatric disorder associated with glutamate dysfunction.

In one embodiment, a compound or composition of the present disclosure is used in the manufacture of a medicament for the treatment of Alzheimer's disease. The compound may be Compound 1.

### Alzheimer's Disease

Despite the millions of individuals afflicted, there remains no disease-modifying therapy for Alzheimer's disease (AD). Without being bound to theory, evidence of Aß peptide accumulation is required for AD diagnosis. Key accompaniments are misfolded Tau (MAPT) accumulation, phosphorylation and spreading, plus microglial and astroglial reactivity. Genetic evidence from dominant early onset AD mutations in APP and presenilins, from Down's syndrome, and from protective APP alleles are all consistent with a causative role for AB. Biomarker studies document AB accumulation two decades prior to symptoms, consistent with a triggering role for AB. However, the temporal disconnection between Aß accumulation and symptoms, as well as the clinical failure of multiple AB-lowering therapies have expanded the focus of AD pathophysiology. Genetic studies implicate microglia in AD risk and progression, so the role of brain innate immunity in symptomatic AD has attracted growing interest. For example, TREM2 signaling has multiple effects on Aß-driven pathology. In addition, classic complement cascade components have been directly implicated in phagocytic synaptic removal in AD, though the basis of synaptic selectivity is not defined.

Crucial for clinical AD progression from pre-symptomatic to mild cognitive impairment (MCI) to dementia is the loss of synapses. Synapse loss was initially documented ultrastructurally at autopsy, though it can now be tracked indirectly with FDG-PET or directly by SV2A PET. PET tracers targeting synaptic vesicle glycoprotein 2A (SV2A), which is ubiquitously expressed in synaptic vesicles, permit quantification of synaptic density from non-invasive scans, facilitating the tracking of neurodegenerative disease progression and drug development. PET imaging with [¹¹C]UCB-J previously demonstrated significantly reduced hippocampal synaptic density of Alzheimer's MCI patients relative to unimpaired controls and was also able to detect drug rescue of synapse loss in a mouse AD model (APPswe/PS1ΔE9; henceforth, APP/PS1). The second generation tracer, [¹⁸F]SynVesT-1, has a longer half-life and higher resolution, with greater potential for clinical translation, and was able to discern differences in synaptic density between APP/PS1 mice and control littermates.

With regard to biochemical events at failing AD synapses, the cellular prion protein (PrP^{C}) was identified as a high affinity receptor for Aß oligomers (Aßo) through the only reported unbiased genome-wide expression cloning screen. PrP^{C} exhibits a unique selectivity for oligomeric Aß. In AD models, Aßo binding to PrP^{C} has a role in synaptic plasticity impairment, learning and memory deficits, and synapse loss. A screen for post-synaptic transmembrane proteins linking Aßo and PrP^{C} to intraneuronal signaling identified neuronal metabotropic glutamate receptor 5 (mGluR5) as key for linking to Fyn and Pyk2 (PTK2B). Importantly, these kinases are linked to Tau and implicated in AD risk. Genetic loss and pharmacological inhibition studies have demonstrated that reduced mGluR5 activity alleviates synaptic and memory deficits in multiple AD models. However, the molecular and cellular basis for mGluR5's role in AD synapse loss, including neuro-glial interactions and complement-driven synaptic phagocytosis, remains unclear.

Allosteric mGluR5 modulators have been subdivided as positive (PAMs), negative (NAMs) and silent (SAMs). PAMs enhance and NAMs suppress glutamate-induced G-protein-mediated Ca²⁺ mobilization and/or shift glutamate efficacy. Multiple NAMs reduce both physiological glutamate signaling and Aßo-PrP^{C}-dependent synaptic deficits. As a dose-limiting side-effect, blockade of glutamate at mGluR5 with a NAM impairs learning and memory independently of AD. Surprisingly, Compound 1 does not alter basal or glutamate signaling but does inhibit the PrP^{C}-mGluR5 interaction to prevent pathological Aßo signaling. Compound 1 greatly expands the potential therapeutic window for mGluR5 as a disease-modifying AD target.

In some embodiments, Compound 1 is used to regulate the expression of neuronal and glial genes, regulate neuro-immune interaction in AD synapse loss, restore synaptic density, and prevent synaptic localization of C1q without altering total C1q levels or overall gliosis.

In some embodiments, Compound 1 is used to increase synaptic density, slow down the loss of synaptic density, reduce the loss of synaptic density, increase pre- and post-synaptic marker levels (SV2A and PSD-95), recover the loss of synaptic markers (SV2A and PSD-95), prevent seizure, inhibit the human transporters P-glycoprotein (P-gp), organic ion transporting polypeptide (OATP)1B1 and OATP1B3. In some embodiments, Compound 1, is used to regulate cerebrospinal fluid (CSF) or plasma biomarkers, such as but not limited to total Tau protein levels, phospho-tau protein levels, SNAP-24, neurofilament light chain, GAP-43, synaptotagmin-1, alpha-synuclein (including phosphorylated versions), and neurogranin. In some embodiments, Compound 1 is used to regulate complement activation biomarkers.

In some embodiments, patients who receive Compound 1 have changes in fluorodeoxyglucose (FDG)-positron emission tomography (PET) imaging, synaptic vesicle glycoprotein 2A (SV2A)-PET imaging, metabotropic glutamate receptor subtype 5 (mGluR5)-PET imaging, Tau PET imaging, amyloid beta PET imaging, electroencephalography (EEG) activities, functional magnetic resonance imaging and/or volumetric magnetic resonance imaging.

In some embodiments, the patient is diagnosed with either amnestic mild cognitive impairment (aMCI) or mild dementia due to AD, e.g., based on the following:

| Mild dementia due to AD | aMCI due to AD |
|---|---|
| (i) National Institute on Aging | (i) Subjective memory complaint preferably corroborated by an informant |
| (NIA)-Alzheimer's | (ii) aMCI as evidenced by abnormal memory function documented by |
| Association core clinical | scoring 1.5 standard deviations (SD) below the education adjusted cutoff |
| criteria for dementia due to | on the Logical Memory II subscale (Delayed Paragraph Recall) from the |
| probable AD | Wechsler Memory Scale - Revised (maximum score is 25): ≤8 for 16 or |
| (ii) Mini Mental Status Exam | more years of education; ≤4 for 8-15 years of education; ≤2 for 0-7 years |
| (MMSE) score between 18 | of education |
| and 26 (inclusive) | (iii) Normal activities of daily living |
| (iii) Clinical Dementia Rating | (iv) CDR global score of 0.5. |
| Scale (CDR) global score of | |
| 0.5 or 1 | |

### Combination Therapies

In some embodiments, the present invention provides a method of treating a disease or disorder described herein, including administering a compound or composition of the present disclosure in combination with one or more additional active agents or therapies. Suitable pharmaceutical agents or therapies that may be used in combination with the compound of the present disclosure include but not limited to anti-amyloid immunotherapies, anti-tau immunotherapies, active agents aimed at reducing amyloid beta levels in any form, active agents that those specifically aimed at blocking amyloid-beta oligomer toxicity, microglial inflammation targeted therapies, acetylcholinesterase inhibitors, and NMDA receptor antagonists (such as but not limited to donepezil and memantine).

The compound or composition of the present disclosure and the additional active agent(s) may be administered simultaneously, sequentially, or at any order. The compound or composition of the present disclosure and the additional active agent(s) may be administered at different dosages, with different dosing frequencies, or via different routes, whichever is suitable.

### IV. Dosing and Administration

The present disclosure encompasses the delivery of a compound or composition of the present disclosure for any therapeutic, prophylactic, pharmaceutical, diagnostic or imaging use by any appropriate route taking into consideration likely advances in the sciences of drug delivery.

A compound or composition of the present disclosure may be administered by any route which results in a therapeutically effective outcome. These include, but are not limited to oral, intravenous (into a vein), intrathecal (into the spinal canal or into the subarachnoid space to reach the CSF), intraparenchymal (into the brain parenchyma), in ear drops, nasal aerosol or inhalation. In specific embodiments, a compound or composition may be administered in a way which allows it to cross the blood-brain barrier, vascular barrier, or other epithelial barrier.

In some embodiments, a compound or compositions of the present disclosure may be orally administered in any orally acceptable dosage form including, but not limited to, capsules, tablets, and aqueous suspensions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are administered orally, the active ingredient may be combined with emulsifying and suspending agents. If desired, certain sweetening and/or flavoring and/or coloring agents may be added.

In some embodiments, a compound or composition of the present disclosure may be formulated to be administered to the CNS by routes known in the art such as, but not limited to, direct intraparenchymal administration, intrathecal delivery and intracerebroventricular infusion.

### Dosage Forms

A pharmaceutical composition described herein can be formulated into a dosage form described herein, such as a capsule, tablet, aqueous suspension or solution, topical, intranasal, intratracheal, or injectable (e.g., intravenous, intraocular, intravitreal, intramuscular, intracardiac, intraperitoneal, subcutaneous). It will be understood that the total daily usage of a composition of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, a composition in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, from about 25 mg/kg to about 50 mg/kg, from about 50 mg/kg to about 100 mg/kg, from about 100 mg/kg to about 125 mg/kg, from about 125 mg/kg to about 150 mg/kg, from about 150 mg/to about 175 mg/kg, from about 175 mg/kg to about 200 mg/kg, from about 200 mg/kg to about 250 mg/kg of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In some embodiments, a compound or composition of the present disclosure is administered by continuous infusion.

As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose.

The administration of a compound or composition of the present disclosure can be used as a chronic or acute therapy. The amount of drug that may be combined with the carrier to produce a single dosage form will vary depending upon the host treated and the particular mode of administration. A typical preparation will contain from about 5% to about 95% active compound (w/w).

Upon improvement of a patient's condition, a maintenance dose of a compound, composition, or combination of the present disclosure may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

As the skilled artisan will appreciate, lower or higher doses than those recited above may be required. Specific dosage and treatment regimens for any particular patient will depend upon a variety of factors, including the activity of the specific compound employed, the age, body weight, general health status, gender, diet, time of administration, rate of excretion, drug combination, the severity and course of an infection, the patient's disposition to the infection and the judgment of the treating physician.

In some embodiments, the subjects are treated with about 10 mg, about 40 mg, about 70 mg, about 100 mg, about 150 mg, or about 200 mg of Compound 1. In some embodiments, the subjects are treated once or twice a day. In a non-limiting example, the subjects are treated with between about 75 mg to about 125 mg of Compound 1 twice a day. In another non-limiting example, the subjects are treated with at least 200 mg of Compound 1 once a day.

In some embodiments, a compound or composition of the present disclosure are administered in capsules via oral routes. In some embodiments, the capsules contain about 5 mg, about 25 mg, about 50 mg, about 75 mg, or about 100 mg of the compound. In some embodiments, the capsules further includes lactose monohydrate, croscarmellose sodium, and/or magnesium stearate. The lactose monohydrate may be spray dried. The croscarmellose sodium and magnesium stearate are in extra-granular form. In some embodiments, the concentration of the compound in the capsules is between about 1% (w/w) to about 50% (w/w), such as between about 1% (w/w) to about 5% (w/w), between about 6% (w/w) to about 10% (w/w), between about 11% (w/w) to about 20% (w/w), between about 21% (w/w) to about 30% (w/w), between about 31% (w/w) to about 40% (w/w), or between about 41% (w/w) to about 50% (w/w). In one embodiment, the capsule has about 5 mg Compound 1, and Compound 1 has a concentration of about 5% (w/w) in the capsule. In another embodiment, the capsule has about 50 mg or 100 mg of Compound 1, and Compound 1 has a concentration of about 25% (w/w) in the capsule.

In some embodiments, the capsules are stored at 25° C./60% relative humidity (RH). The compound in the capsules is stable for at least 30 months, and the capsules are stable for at least 1 month, under accelerated and long-term stability conditions.

### V. Kits and Devices

The disclosure also provides a variety of kits and devices for conveniently and/or effectively carrying out methods of the present disclosure. Typically, kits will include sufficient amounts and/or numbers of components to allow a user to perform multiple treatments of a subject(s) and/or to perform multiple experiments.

In one embodiment, the present disclosure provides kits for treating CNS disorders, including a compound or composition of the present disclosure, optionally in combination with any other active agents.

The kit may further include packaging and instructions and/or a delivery agent to form a formulation composition. The delivery agent may include a saline, a buffered solution, or any delivery agent disclosed herein. The amount of each component may be varied to enable consistent, reproducible higher concentration saline or simple buffer formulations. The components may also be varied in order to increase the stability of the compounds in the buffer solution over a period of time and/or under a variety of conditions.

The present disclosure provides for devices which may incorporate a compound or composition of the present disclosure. These devices contain in a stable formulation available to be immediately delivered to a subject in need thereof, such as a human patient. In some embodiments, the subject has a CNS disorder.

Non-limiting examples of the devices include a pump, a catheter, a needle, a transdermal patch, a pressurized olfactory delivery device, iontophoresis devices, multi-layered microfluidic devices. The devices may be employed to deliver a compound or composition of the present disclosure according to single, multi- or split-dosing regiments. The devices may be employed to deliver a compound or compositions of the present disclosure across biological tissue, intradermal, subcutaneously, or intramuscularly.

### VI. Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

The abbreviations used herein have their conventional meaning within the scientific arts. The chemical elements are identified in accordance with the Periodic Table of the Elements, CAS version, Handbook of Chemistry and Physics, 75th Ed. Additionally, general principles of organic chemistry are described in M. Loudon, Organic Chemistry, 5th Ed., Roberts and Company, Greenwood Village, Colo.: 2009; and M. B. Smith, March's Advanced Organic Chemistry: Reactions, Mechanisms and Structure, 7th Ed., John Wiley & Sons, Hoboken: 2013, the entire contents of which are hereby incorporated by reference.

As used herein, the term “about” means+/−10% of the recited value.

The term “compound”, as used herein, is meant to include all stereoisomers, geometric isomers, tautomers, and isotopes of the structures depicted.

The term “substantially purified” refers to the state of being free of other, dissimilar compounds and impurities with which the crystalline forms of the invention are normally associated in its natural state, so that the “substantially purified” crystalline form is at least 95% of the mass, by weight, of a given sample.

The terms “subject” or “patient”, as used herein, refer to any organism to which the particles may be administered, e.g., for experimental, therapeutic, diagnostic, and/or prophylactic purposes. Typical subjects include animals (e.g., mammals such as mice, rats, rabbits, guinea pigs, cattle, pigs, sheep, horses, dogs, cats, hamsters, lamas, non-human primates, and humans).

The terms “treating” or “preventing”, as used herein, can include preventing a disease, disorder or condition from occurring in an animal that may be predisposed to the disease, disorder and/or condition but has not yet been diagnosed as having the disease, disorder or condition; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

The terms “managing” or “maintaining”, as used herein, can refer to reducing the symptom(s) of a disease, reducing the severity of symptom(s) of the disease, or preventing the symptom(s) of the disease from getting worse.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease, disorder or condition in the enhancement of desirable physical or mental development and conditions in an animal, e.g., a human.

The term “modulation” is art-recognized and refers to up regulation (i.e., activation or stimulation), down regulation (i.e., inhibition or suppression) of a response, or the two in combination or apart. The modulation is generally compared to a baseline or reference that can be internal or external to the treated entity.

The terms “sufficient” and “effective”, as used interchangeably herein, refer to an amount (e.g., mass, volume, dosage, concentration, and/or time period) needed to achieve one or more desired result(s). A “therapeutically effective amount” is at least the minimum concentration required to affect a measurable improvement or prevention of at least one symptom or a particular condition or disorder, to affect a measurable enhancement of life expectancy, or to generally improve patient quality of life. The therapeutically effective amount is thus dependent upon the specific biologically active molecule and the specific condition or disorder to be treated. Therapeutically effective amounts of many active agents, such as antibodies, are known in the art. The therapeutically effective amounts of compounds and compositions described herein, e.g., for treating specific disorders may be determined by techniques that are well within the craft of a skilled artisan, such as a physician.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The term “pharmaceutically acceptable”, as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “pharmaceutically acceptable salt(s)” refers to salts of acidic or basic groups that may be present in compounds used in the present compositions.

The term “substantially purified” refers to a purity of a compound of at least 90%, e.g., about 90, about 95, or about 99%, relative to other forms of that compound.

The details of one or more embodiments of the disclosures are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### EXAMPLES

### Example 1. Polymorph Screening and Characterization

### Crystallization Development

The polymorph study protocol involved the use of a selection of solvents in which the solubility of Compound 1 was initially evaluated. Four typologies of experiments were then set up: temperature cycling, evaporative, long-term slurries and vapor diffusion. The number of solvents, solvent volumes and amount of material to be used in each experiment was based on solubility values and the kind of experiment.

Approximate solubility (visual) of Compound 1 was assessed in a list of solvents selected based on class diversity including solvent/water mixtures (Table 5). Compound 1 (5.0±0.2 mg) was weighted in vials and known aliquots of solvent were progressively added at 20° C. until a clear solution was visually observed; magnetic stirring was applied. The final solutions were stirred at room temperature overnight to evaluate any re-crystallization phenomena.

TABLE 5
Approximate solubility of Compound 1

| Solvent | Approx. solubility (mg/mL) |
|---|---|
| acetone | >100 |
| methyl ethyl ketone (MEK) | >100 |
| methyl isobutyl ketone (MIK) | >100 |
| acetonitrile | 50 < S < 100 |
| benzonitrile | >100 |
| toluene | 25 < S < 30 |
| dichloromethane | >100 |
| methyl tert-butyl ether (MTBE) | 17 < S < 25 |
| tetrahydrofuran (THF) | >100 |
| 2-methyltetrahydrofuran (MeTHF) | >100 |
| ethyl acetate | >100 |
| i-propyl acetate | 50 < S < 100 |
| t-Butyl acetate | 25 < S < 30 |
| ethanol | >100 |
| isopropyl alcohol (IPA) | 25 < S < 30 |
| 2-butanol | 50 < S < 100 |
| heptane | <2 |
| water | <2 |
| acetone/water 90:10 | >100 |
| acetone/water 50:50 | S~5 |
| 2-propanol/water 90:10 | 30 < S < 50 |
| 2-propanol/water 80:20 | 50 < S < 100 |
| 2-propanol/water 50:50 | S~5 |

In general, the compound showed medium/high solubility in most of the screened solvents. Only heptane and water acted as anti-solvents. Because of the high solubility, solvents for the following experiments were selected depending on the solvent properties (e.g. boiling points, miscibility) and solubility.

A total of 38 samples were prepared. The experiments involved in the protocol were temperature cycling, evaporative, long-term slurries and vapor diffusion experiments.

### Temperature Cycling

Slurries of Compound 1 were prepared in eight solvent systems with solubility <30 mg/mL. In Integrity workstation, the slurries were subjected to six temperature cycles under magnetic stirring (650 rpm). Each cycle included heating/cooling ramps at rate of 2° C./min and step duration at 20° C. and 40° C. of 110 minutes. The presence of solid at hot conditions was ensured by visual check. Solid samples were isolated by filtration on 20 μm PTFE filter syringes and dried under vacuum at 20° C. for about 2 hours prior to be submitted to XRPD analysis.

### Evaporative Experiments

Twelve organic solvents with boiling points lower than 110° C. and in which solubility was greater than 15 mg/mL were selected for evaporative experiments.

Almost saturated solutions of Compound 1 were prepared in the selected solvent systems and stirred at room temperature for about 1 hour to maximize dissolution equilibria. The mixtures were filtered into new vials through 0.45 μm PTFE filters. The resulting clear solutions were evaporated in a dry-box under inert atmosphere, e.g., nitrogen gas (open vials, no stirring applied).

### Long-Term Slurries

Slurries of Compound 1 were prepared in eight solvent systems that were selected considering solubility <30 mg/mL. The slurries were placed in a thermostatic block at 20° C. and stirred over two weeks.

The solvate Form B (i.e., Solvate S1) was prepared using evaporative experiments. An almost saturated solution of Compound 1 was prepared in isopropyl alcohol and stirred at room temperature for about 1 hour to maximize dissolution equilibria. The mixture was filtered into new vials through 0.45 p.m PTFE filters (open vials, no stirring applied). Within one week, almost all isopropyl alcohol evaporated. The residues were dried in vacuum oven at 20° C. for 1 hour.

The solvate Form C (i.e., Solvate S2) was prepared using a long-term slurry in isopropyl alcohol. The slurry was placed in a thermostatic block at 20° C. and stirred over two weeks. Solid samples were isolated by filtration on 20 p.m PTFE filter syringes and dried under vacuum for about 2 hours.

The solvate Form C (i.e., Solvate S2) was also prepared using vapor diffusion using isopropyl alcohol and pentane as an anti-solvent. A concentrated solution of Compound 1 was prepared in isopropyl alcohol and stirred at room temperature for about 1 hour to maximize dissolution equilibria. The mixture was filtered through 0.45 p.m PTFE filters into a new vial and placed without a cap into outer vials containing pentane. The system was sealed, stored at room temperature, stirred, and periodically checked for solid formation. Solids of Form C only formed when the mixture was stirred. Solids were isolated by supernatant removal and dried for at least 30 minutes in vacuum oven at room temperature.

A summary of the results is showin in the table below.

TABLE 6
Results with various solvents with different process

| | Temper- | | | |
|---|---|---|---|---|
| | ature | Evapo- | Long term | Vapour |
| Solvent system | cycling | rative | Slurries | diffusion |
| acetone | | amorphous | | Form A |
| methyl ethyl ketone | | | | Form A |
| methyl isobutyl | | | | Form A |
| ketone | | | | |
| acetonitrile | | amorphous | | |
| toluene | Form A | Form A | Form A | Form A |
| dichloromethane | | amorphous | | |
| methyl tert-butyl | Form A | Form A | Form A | Form A |
| ether | | | | |
| tetrahydrofuran | | amorphous | | Form A |
| 2- | | Form A | | |
| methyltetrahydrofuran | | | | |
| ethyl acetate | | Form A | | Form A |
| i-propyl acetate | | Form A | | Form A |
| t-Butyl acetate | Form A | | Form A | |
| methanol | | amorphous | | |
| 2-propanol | Form A | Solvate S1 | Solvate S2 | Solvate S2 |
| | | (Form B) | (Form C) | (Form C) |
| 2-butanol | | Form A | | Form A |
| heptane | Form A | | Form A | |
| water | Form A | | Form A | |
| acetone/water | Form A | | Form A | |
| 50:50 | | | | |
| 2-propanol/water | Form A | | Form A | |
| 50:50 | | | | |

Further studies were carried out and found Form A is an anhydrous (AH) form and was isolated using a mixture of EtOAc and heptane that provided crystalline material with significant yield and high purity grade.

Solubility data were generated applying the following protocol: slurries of Compound 1 were prepared and allowed to equilibrate at 20° C. under stirring for about 18 hours. To determine the concentration in supernatant, about 0.1 mL of slurry was sampled with syringe, filtered, weighed, diluted with MeCN, and injected into an HPLC. The slurries were heated for about 3 hours at 45° C., then, sampling was repeated to get solubility data at hot conditions. Samples were cooled to 20° C., and solid residues were isolated by filtration on 20-μm PTFE filter, dried under vacuum at 40° C., and submitted to XRPD analysis. Compound 1 peak area for each sample was recorded, and solubility data were calculated using previously determined response factor (245 nm, 1460 mAU*sec/mg/mL, Table 7). The data were combined and visualized on chart with the solubility as function of the percentage of heptane by volume. Compound 1 showed high solubility in neat EtOAc as well as in the presence of relative high percentages of heptane (10-20). Solubility smoothly decreases with the increase in heptane amount. All solid residues showed physical form concordant with Form A.

TABLE 7
Compound 1 solubility data
Compound 1 solubility data (mg/mL) - EtOAc/heptane mixtures (% v/v)

| EtOAc | Heptane | 20° C. | 45° C. | XRPD |
|---|---|---|---|---|
| 100 | 0 | 115 | 187 | Form A |
| 90 | 10 | 95 | 177 | Form A |
| 80 | 20 | 69 | 124 | Form A |
| 70 | 30 | 49 | 85 | Form A |
| 60 | 40 | 33 | 61 | Form A |
| 50 | 50 | 20 | 34 | Form A |
| 40 | 60 | 11 | 17 | Form A |
| 32 | 68 | 6 | 10 | Form A |
| 20 | 80 | 2 | 3 | Form A |
| 0 | 100 | 0 | 0 | Form A |

Based on the solubility data and, in order to identify the proper operating conditions/seeding points, metastable zone widths in EtOAc/heptane in the ratios 80:20, 70:30 and 60:40 were tested.

Metastable zone widths were generated using Electrothermal Integrity equipment coupled with IR turbidity probes. Samples with different amount of material were weighed in vial and diluted with 1 mL of the proper solvent mixture. The screened concentrations were in the range 115-215 mg/mL considering the contribution of the solid that was estimated to be corresponding to about 1 volume. A step/plateau temperature program was set: heating rate of 0.1° C./min, step of 1° C. and plateau duration of 1 minute. The step up heating was set at 78° C. (the maximum temperature allowed by the solvent component with the lowest boiling point); with the same parameters, a step down cooling was applied. Vials were monitored by turbidity probes to obtain dissolution temperature (over heating ramp) and self-crystallization temperature (over cooling ramp) for each concentration.

All solvent compositions provided a regular dissolution profile with good fitting to the solubility data previously collected. Selected samples were isolated by filtration and analysed by XRPD: consistency with Form A was confirmed. Overall, EtOAc/heptane 80:20 ratio appeared to be a good option for dissolution, while composition 70:30 was preferred as seeding point. The latter, in fact, offered a practical range of concentrations where it was possible to drive comfortably the initial solution toward the supersaturated region. On the other side, the MSZW for 60:40 solvents ratio was deemed too narrow and risky from an operative point of view.

### Characterization

### XRPD

The XRPD spectra were collected in transmission mode on a Panalytical X'Pert Pro or Empyrean instrument with X'Celerator detector using a standard method. The samples were irradiated with Cu Kα X rays. The data were evaluated using the Panalytical Data Viewer software. The details of the standard screening data collection method are listed below. A representative XRPD graph for AH Form A is shown in FIG. 3. A representative XRPD graph for Form B is shown in FIG. 5. A representative XRPD graph for Form C is shown in FIGS. 7 and 9.

| | |
|---|---|
| Start Position [°2θ] | 2.0104 |
| End Position [°2θ] | 44.9864 |
| Step Size [°2θ] | 0.0170 |
| Scan Step Time [s] | 59.6900 |
| Scan Type | Continuous |
| PSD Mode | Scanning |
| PSD Length [°2θ] | 2.12 |
| Offset [°2θ] | 0.0000 |
| Divergence Slit Type | Fixed |
| Divergence Slit Size [°] | 0.4354 |
| Specimen Length [mm] | 10.00 |
| Measurement Temperature [° C.] | 25.00 |
| Anode Material | Cu |
| Intended Wavelength Type | K-Alpha |
| K-Alpha1 [Å] | 1.54060 |
| K-Alpha2 [Å] | 1.54443 |
| K-Beta [Å] | 1.39225 |
| K-A2/K-A1 Ratio | 0.50000 |
| Generator Settings | 40 mA, 40 kV |
| Diffractometer Type | 0000000011016732 |
| Diffractometer Number | 0 |
| Goniometer Radius [mm] | 240.00 |
| Dist. Focus-Diverg. Slit [mm] | 100.00 |
| Incident Beam Monochromator | No |
| Spinning | Yes |

### TGA

The TGA analyses were run on a TA Q5000 instrument. TGA method details are listed below. A representative graph for AH Form A is shown in FIG. 4. A representative graph for Form B is shown in FIG. 6. A representative graph for Form C is shown in FIGS. 8 and 10.

| Instrumental parameter | Value |
|---|---|
| Balance purge gas [mL/min] | 10 |
| Sample purge gas [mL/min] | 25 |
| Gas | Nitrogen |
| Temperature- Time-Rate | Typically from room temperature to 250/ |
| | 350° C. at 10° C./min |
| Typical sample amount [mg] | Usually from 2 mg to 20 mg |
| Pan [Pt/Al] | Sealed Aluminium (punched) |

### DSC

The DSC analyses were run on a TA Q5000 instrument. DSC method details are listed below. A representative graph for AH Form A is shown in FIG. 4. A representative graph for Form B is shown in FIG. 6. A representative graph for Form C is shown in FIGS. 8 and 10.

| Instrumental parameter | Value |
|---|---|
| Balance purge gas [mL/min] | 10 |
| Sample purge gas [mL/min] | 25 |
| Gas | Nitrogen |
| Temperature-Time-Rate | Typically from room temperature to 250/ |
| | 300° C. at 10° C./min |
| Typical sample amount [mg] | Usually from 1 mg to 5 mg |
| Pan [Pt/Al] | Sealed Aluminium (punched) |

### Example 2. Intravenous and Oral Investigative Cross-Over Pharmacokinetic Study in the Male CD Rat

This study assessed the PK of Compound 1 AH Form A in male Sprague Dawley rats (3/group) following single IV (intravenous) administration at 1 mg/kg as a solution in 2.5% N-Methyl-2-pyrrolidone (NMP)+1% Solutol HS15 in water or a PO (oral) administration of 3 formulations at 5 and 50 mg/kg, administered to the same animals in a cross over with 7 days recovery. For PO administration the compound was formulated as a nanomilled suspension (Formulation A) or as 2 different solutions (Formulation B or C).

Serial plasma samples were collected at intervals to 24 h post dose and the level of Compound 1 determined in each plasma sample by LC-MS/MS. Pharmacokinetic parameters were determined and are shown in Table 8.

TABLE 8
Plasma pharmacokinetic parameters of Compound 1 following
single intravenous and oral administration to male rats

| Dose | | | Tₘₐₓ | Cₘₐₓ | AUCₗₐₛₜ | |
|---|---|---|---|---|---|---|
| (mg/kg) | Route | Formulation | (h) | (ng/mL) | (ng · h/mL) | F % |
| 1 | IV | | NA | 595 | 2910¹ | NA |
| 5 | PO | A | 8.00 | 1130 | 9140 | 62.8 |
| 50 | PO | A | 6.00 | 8850 | 168000 | 100 |
| 5 | PO | B | 8.00 | 2740 | 26800 | 100 |
| 50 | PO | B | 8.00 | 7980 | 140000 | 96.5 |
| 5 | PO | C | 6.00 | 1310 | 14200 | 97.7 |
| 50 | PO | C | 8.00 | 7210 | 128000 | 87.6 |

NA—Not applicable.
¹AUC_{inf} reported for the IV dose and used to determine the F %.
A - Nanomilled suspension (10% (w/w) Compound 1) in 2% (w/w) of HPMC 603 and 0.5% (w/w) SLS in MilliQ water.
B - 40% PEG 400, 20% Transcutol, 10% Vit E TPGS, 0.5% Poloxamer 407 in water
C - 5% DMSO, 0.5% HPMC in water

Following each PO dose there was an initial plasma peak at 0.5 to 2 h post dose, levels then declined and there was a second higher peak at 6-8 h post dose. The F % calculated from the AUC_{inf} for the IV and AUCₗₐₛₜ for PO doses was high for all formulations.

Formulation A showed PO exposure (as AUCₗₐₛₜ) 0.6 and 1.3 fold higher than values of the standard formulation C at 5 and 50 mg/kg, respectively.

Formulation B showed PO exposure (as AUCₗₐₛₜ) 1.8 and 1.1 fold higher than values of the standard formulation C at 5 and 50 mg/kg, respectively.

At 50 mg/kg, the highest systemic exposure to Compound 1 (AUCₗₐₛₜ, 168000 ng·h/mL) as well as the highest Cₘₐₓ (8847 ng/ml) was observed with the nanosuspension formulation (Form A) and this formulation was used for the 28 day toxicology studies in rat and monkey.

Following the IV dose T_{1/2} was 4.49 h, the V_{dss} was 2.23 L/kg and CL was 5.98 mL/min/kg.

### Example 3. Methods of Preparing Formulations of Compounds

### 1. Nanosuspesnion Composition

Studies were performed to select a suitable nano-suspension composition for AH Form A of Compound 1. Three qualitatively different vehicles were prepared at a scale of 200 g each, in order to verify the feasibility and stability of nano-milled suspension in these media.

Vehicles were prepared in advance by solubilizing the proper amount of polymer and surfactant in purified water (MilliQ®). Complete solubilization of materials was achieved by magnetic stirring. Compound 1 Form A was then added to the prepared vehicle and suspended (homogeneous suspension was achieved approximately within 15 min for all the compositions).

The list of materials is shown in Table 9. Qualitative and quantitative formulation compositions are reported in Table 10.

TABLE 9
List of materials

| MATERIAL | FUNCTION | BRAND NAME |
|---|---|---|
| Compound 1 Form A | Active substance | N/A |
| Kolliphor PS80 | Surfactant | Tween 80 |
| Sodium lauryl sulfate | Surfactant | Kolliphor SLS fine |
| Hypromellose 2910 | Polymer | Pharmacoat 603 |
| Hypromellose 2910 | Polymer | Pharmacoat 606 |
| Yttrium zirconia beads 0.4 mm | Grinding material | N/A |
| Yttrium zirconia beads 0.1 mm | Grinding material | N/A |
| Purified water | Solvent | N/A |

TABLE 10
Qualitative compositions investigated
Suspensions composition

| Batch No. | Composition 1 | | Composition 2 | | Composition 3 | |
|---|---|---|---|---|---|---|
| Material | % (w/w) | g/batch | % (w/w) | g/batch | % (w/w) | g/batch |
| Compound | 10.00 | 20.00 | 10.00 | 20.00 | 10.00 | 20.00 |
| 1 Form A | | | | | | |
| Polysorbate | 2.50 | 5.00 | — | — | — | — |
| 80 | | | | | | |
| HPMC 606 | — | — | 2.00 | 4.00 | — | — |
| HPMC 603 | — | — | — | — | 2.00 | 4.00 |
| SLS | — | — | 0.50 | 1.00 | 0.50 | 1.00 |
| MilliQ | 87.50 | 175.00 | 87.50 | 175.00 | 87.50 | 87.50 |
| Water | | | | | | |
| TOT | 100.00 | 200.00 | 100.00 | 200.00 | 100.00 | 200.00 |

Aiming to investigate the feasibility of the nanomilling process, small scale Dynomill (Dynomill RL) was utilized at this stage. Upon assembling, the milling chamber was filled to 65% of total volume (50 mL) with grinding media beads (equivalent to 32.5 mL of 0.4 mm YTZ milling beads, corresponding to approx. 120 g). The manufactured nano-suspensions were characterized for: Particle size distribution (PSD) by Malvern Mastersizer (LLS);
Particle size distribution by Malvern Zeta sizer (Z-sizer);
Z-potential; X-ray diffraction (XRPD);
Assay/impurities by HPLC; and
Homogeneity by HPLC.

Based on the physical and chemical characterization of the nanomilled compositions, it is possible to conclude:
Composition 2 and composition 3 show similar PSD characteristics (monomodal distribution) with target size reached after 60 minutes of milling, while composition 1 showed bi-modal distribution with presence of particles >1 μm
Composition 2 and composition 3 show better Z-potential values in line with typical stable nano-suspension products (lower values than-25 mV)
XRPD data showed that the nano-milling process does not impact the crystalline form for all the 3 compositions

All three nano-suspension compositions were subjected to stability studies (refrigerated conditions only). The results of 7 days stability were focused on the appearance of the formulations, stirring method and sampling point.

### Informal Stability: 7 Days Timepoint

The three manufactured nano-suspensions were characterized after 7 days, stored at refrigerated conditions. The analyses carried out were:
Particle size distribution by laser light scattering by Malvern Mastersizer;
Z-potential measurement by Malvern Zetasizer;
X-ray pattern diffraction; and
Homogeneity and impurities by HPLC.

Based on the results from physical and chemical characterization, composition 1 was confirmed to be more unstable and not homogeneous.

### Informal Stability: 14 Days

The stability of compositions 2 and 3 were assessed through 14 days storing under refrigerated conditions. The following tests were carried out:
Particle size distribution by laser light scattering by Malvern Mastersizer;
Z-potential measurement by Malvern Zetasizer; X-ray pattern diffraction; and Homogeneity and impurities by HPLC.

Based on the results from physical and chemical characterization, both compositions 2 and 3 can be considered stable for 14 days under refrigerated conditions. However, composition 3 exhibited a more desirable Z-potential value.

Based on this consideration, the following activities were carried out by using the following composition:

TABLE 11
Composition to be progressed

| Material | % (w/w) |
|---|---|
| Compound 1 AH Form A | 10.0 |
| HPMC 603 - Hypromellose 603 | 2.0 |
| SLS - Kolliphor SLS fine | 0.5 |
| Purified water | 87.5 |
| TOTAL | 100.0 |

### 2. Oral Formulation Development

TABLE 12
List of materials

| MATERIAL | FUNCTION | BRAND NAME |
|---|---|---|
| Compound 1 AH Form A | Active substance | N/A |
| Sodium lauryl sulfate | Surfactant | Kolliphor SLS fine |
| Hypromellose 2910 | Polymer | Pharmacoat 603 |
| YTZ (yttria-stabilised zirconia) | Grinding material | N/A |
| beads 0.4 mm | | |
| Yttrium zirconia beads 0.2 mm | Grinding material | N/A |
| Purified water | Solvent | N/A |
| Lactose monohydrate | Carrier | Supertab 11 SD |
| Mannitol | Carrier | Pearlitol 200 SD |
| Magnesium stearate | Lubricant | Ligamed MF-2-V |
| Croscarmellose | Disintegrant | Ac-di-Sol |
| HPMC size 0 Swedish orange | Capsules shell | N/A |
| capsules | | |
| HPMC size 00 Swedish orange | Capsules shell | N/A |
| capsules | | |

The nano-suspension prepared was divided into sublots (four aliquots) and used to investigate top spray granulation. Two water-soluble bulking agents/carriers were tested:
Lactose monohydrate, Supertab 11 SD (spray dried grade)
Mannitol, Pearlitol 200 SD (spray dried grade)

Aiming to achieve a compound-carrier matrix inhibiting crystal growth, a small amount of above-mentioned materials were solubilized in the aliquot of nanomilled suspension to be sprayed (ratio 1:1 with the Compound 1 concentration). Four trials of top spray granulation (2 carriers at 2 drug loads) were carried out as follows:
330 g of nanomilled material (+lactose 11 SD or mannitol 200 SD as bulking agent) were sprayed on 550 g of selected carrier, to obtain approx. a 5% granule DL, suitable drug load to prepare 5 mg capsule strength.
550 g of nanomilled material (+lactose 11 SD or mannitol 200 SD as bulking agent) was sprayed on 100 g of selected carrier, to obtain a approx. a 20% granule DL, suitable drug load to prepare 50 mg and 100 mg capsule strengths.

The following Table 13 shows the composition of the nano-suspension upon dilution with bulking agent:

TABLE 13
Diluted nanosuspension composition

| | Initial | Diluted | | Diluted | |
|---|---|---|---|---|---|
| | nano- | nano-suspension | | nano-suspension | |
| | suspension | for 5% DL | | for 21% | |
| | composition | granules | | DL granules | |
| | % | | % | | % |
| | (w/w) | g/330 g | (w/w) | g/550 g | (w/w) |
| Compound 1 | 10.00 | 30.00 | 9.09 | 50.00 | 9.09 |
| HPMC 603 | 2.00 | 6.00 | 1.82 | 10.00 | 1.82 |
| SLS | 0.50 | 1.50 | 0.45 | 2.50 | 0.45 |
| Water | 87.50 | 262.50 | 79.55 | 437.50 | 79.55 |
| Pearlitol 200SD/ | N/A | 30.00 | 9.09 | 50.00 | 9.09 |
| Lactose 11SD | | | | | |
| as bulking agent | | | | | |
| TOTAL | 100.00 | 330.00 | 100.00 | 550.00 | 100.00 |

Upon addition and solubilization of bulking agent in the aliquot of nano-suspension, carrier was loaded in the granulator bowl to be warmed prior starting the top spray granulation. Table 14 and Table 15 below show the compositions of the four granules prepared:

TABLE 14
Theoretical granules composition with mannitol

| | Capsule 1 | | Capsule 2 | |
|---|---|---|---|---|
| | | % (w/w) | | % (w/w) |
| | g/617.5 g | granule | g/500 g | granule |
| Compound 1 | 30.00 | 4.86 | 50.00 | 21.05 |
| HPMC 603 | 6.00 | 0.97 | 10.00 | 4.21 |
| SLS | 1.50 | 0.24 | 2.50 | 1.05 |
| Mannitol Pearlitol 200SD | 30.00 | 4.86 | 50.00 | 21.05 |
| as bulking agent | | | | |
| Mannitol Pearlitol 200SD | 550.00 | 89.07 | 125.00 | 52.63 |
| as carrier | | | | |
| TOTAL | 617.50 | 100.00 | 237.50 | 100.00 |

TABLE 15
Theoretical granules composition with lactose

| | Capsule 3 | | Capsule 4 | |
|---|---|---|---|---|
| | g/617.5 | % (w/w) | g/500 | % (w/w) |
| | g | granule | g | granule |
| Compound 1 | 30.00 | 4.86 | 50.00 | 21.05 |
| HPMC 603 | 6.00 | 0.97 | 10.00 | 4.21 |
| SLS | 1.50 | 0.24 | 2.50 | 1.05 |
| Lactose 11SD as bulking | 30.00 | 4.86 | 50.00 | 21.05 |
| agent | | | | |
| Lactose 11SD as carrier | 550.00 | 89.07 | 125.00 | 52.63 |
| TOTAL | 617.50 | 100.00 | 237.50 | 100.00 |

Four top spray granulations were carried out in order to investigate the process feasibility and produce enough material for the sub-sequent capsules preparation. The resulting granules (5% and 20% DLs), visually appeared differently: coarse agglomerates for both lactose and mannitol based granules at 20% DL, while very fine agglomerates appeared the 5% granule DLs.

As general observation for the following manufacturing batches, these feasibility trials were carried out with a sub-optimal batch scale in particular for the 20% DL (due to low amount of carrier), while slight adherence of fine particles to granulator walls were observed for both drug loads. Adjustments in batch scale and feed rate were then done with the following batches.

The four prepared granules were characterized for: Loss on drying (LOD) (IPC); Particle size distribution (PSD); X-ray pattern diffraction (XRPD); Particle size upon reconstitution; XRPD of reconstituted; Bulk and tapped densities; Granule homogeneity; and Impurities profile.

From process ability point of view, the lactose composition has better properties compared to mannitol (powder blend movement in the granulator chamber). Moreover, granules at 21% DL with lactose were the only composition able to reconstitute to the input nano-suspension PSD.

Based on these considerations, lactose is advantageous as a carrier.

Extra-granular excipients were added to the four granule batches prior to manual capsule filling. At this stage, 4% (w/w) of disintegrant (Croscarmellose, Ac-di-Sol) and 1% (w/w) of lubricant (magnesium stearate) were added to each granule batch.

The equipment used for blending the granule with extra granular material was a low shear blender (Pharmatec) equipped with proper bowl size (1 L bowl for 20% (w/w) granules and 2 L bowl for the 5% (w/w) granules).

Disintegrant and lubricant were sieved through a 500 microns screen directly into the bowl containing the granule and blended respectively for 10 minutes at 17 rpm and for 3 minutes at 17 rpm.

Two drug loads were prepared for each carrier used, in the specific:
Final blends at 4.62% were then used to prepare 5 mg capsule strength.
Final blends at 20.00% were then used to prepare both the 50 mg and 100 mg capsule strengths.

The final blends composition as % (w/w) is reported in Table 16:

TABLE 16
Final blend compositions

| | | | | |
|---|---|---|---|---|
| | Mannitol | Mannitol | Lactose | Lactose |
| Carrier | % (w/w) | % (w/w) | % (w/w) | % (w/w) |

Intra-granular phase

| | | | | |
|---|---|---|---|---|
| Compound 1 Form A | 4.62 | 20.00 | 4.62 | 20.00 |
| HPMC | 0.92 | 4.00 | 0.92 | 4.00 |
| SLS | 0.23 | 1.00 | 0.23 | 1.00 |
| Bulking agent | 4.62 | 20.00 | 4.62 | 20.00 |
| Carrier | 84.62 | 50.00 | 84.62 | 50.00 |

Extra-granular phase

| | | | | |
|---|---|---|---|---|
| Croscarmellose | 4.00 | 4.00 | 4.00 | 4.00 |
| Magnesium stearate | 1.00 | 1.00 | 1.00 | 1.00 |
| TOTAL | 100.00 | 100.00 | 100.00 | 100.00 |

### Investigating the Bulking Agent

1,800 g of nanomilled material (without bulking agent) was sprayed on 500 g of Lactose 11 SD (carrier), to obtain approximately 24% granule DL. Table 17 below shows the composition of granules prepared:

TABLE 17
Granule composition without bulking agent

Granule composition

| | % (w/w) | g/batch |
|---|---|---|
| Compound 1 | 24.83 | 180.00 |
| Hypromellose 603 | 4.97 | 36.00 |
| Sodium Lauryl | 1.24 | 9.00 |
| Sulphate | | |

| | |
|---|---|
| Water | Removed during drying |

| | | |
|---|---|---|
| Lactose 11SD | 68.97 | 500.00 |
| as carrier | | |
| TOTAL | 100.00 | 725.00 |

From processability point of view, the larger amount of carrier (500 g) suited the granulator bowl capacity, allowing a proper movement of carrier during heating and granulation: upon pre-heating the granulator and carrier, the spraying was started with very low feed rate (approx. 2 g/min) then increased during the process until reaching approx. 12 g/min, by keeping the product temperature of 28-35° C. Granule manufactured appeared fine but visually granulated, with a final yield of 92.14% (669 g of granule). Particle size distribution (by LLS and sieve analysis)

In terms of PSD, the batch shows a tight mono modal size distribution as per previous example, but a smaller ×90 is observed (340 microns vs 500 microns from feasibility). The values detected by laser light scattering are consistent with sieve analysis result.

Exemplary PSD for nanosuspensions are shown in Table 18.

TABLE 18
PSD characterization of the granule composition. The reported
values are averaged from three runs of analysis

| | Granule composition | | |
|---|---|---|---|
| | Run 1 | Run 2 | Run 3 |
| x10 | 0.070 | 0.070 | 0.070 |
| (microns) | | | |
| x50 | 0.135 | 0.134 | 0.134 |
| (microns) | | | |
| x90 | 0.301 | 0.287 | 0.288 |
| (microns) | | | |

### X-Ray Pattern Diffraction (XRPD)

The XRPD of the 20% DL granule (light green trace) shows the presence of Compound 1.

### Particle Size Distribution Upon Reconstitution

Manufactured granule was tested for reconstitution down to nano range. The reconstitution kinetic is lower compared to lactose composition manufactured during the prior example: the faster kinetics previously observed may be attributed to the presence of bulking agent which acts as “stabilizer” and “steric inhibitor” between compound-compound particles, preventing the formation of agglomerates. Despite this, a portion of compound particles was freely reconstituted in the same nano-range of input nanosuspension. Bulk and tapped density upon reconstitution.

The composition showed an improvement in the flow properties compared to the prior example, as confirmed by “Hausner ratio” index.

### Granule Homogeneity

The granules were checked for homogeneity. Six samples were taken from the bulk. As reported in the table below, good potency is obtained for the granule with an average of 100.76% of the theoretical drug load in contrast to the lower mean average detected during feasibility trial (87% (w/w)), confirming the impact of the unsuitable batch size at that stage. Slightly higher RSD is observed, however within limits of acceptability (RSD <5%).

No impurities were detected (below 0.05%).

### Capsule Filling for Dissolution Assessment

Dissolution test was carried out on six manually filled capsules prepared at 100 mg strength with the granules, in order to understand if the absence of bulking agent has an impact on compound release. Dissolution test confirmed the absence of bulking agent improved the compound release. Even if quite high variability is still present between individual capsules (as previously observed), release of more than 90% is achieved for all samples at 60 minutes.

### Example 4. In Vitro Studies

### In Vitro Inhibition of the Human Transporters P-Gp, OATP1B1, OATP1B3 and BSEP

The in vitro potential for Compound 1 Form A to be an inhibitor of the human transporters P-glycoprotein (P-gp), organic ion transporting polypeptide (OATP) 1B1 and OATP1B3 was assessed in various in vitro cell test systems, and of BSEP in membrane vesicles.

A decrease in the transporter-mediated activity of probe substrate in the presence of a positive control inhibitor, determined from incubations run alongside test compound, confirmed that the in vitro test systems were capable of identifying transporter inhibitors.

Under the current assay conditions, Compound 1 Form A was an inhibitor of probe substrate transport mediated via P-gp, OATP1B1, OATP1B3 and BSEP, with IC50 of 27.7, 6.74, 32.0 and 13.9 μM, respectively.

### In Vitro CYP450 Reaction Phenotyping Study in Supersomes™ Over-Expressing Human CYP Enzymes

The CYPP450 enzymes involved in the Phase I metabolism of Compound 1 Form A were investigated in vitro. Compound 1 Form A was metabolized by CYP1A2, CYP2D6 and CYP3A4 and with T1/2 values of 64, 14, and 176 min, respectively, and CLint of 0.261, 0.984 and 0.0786 μL/min/pmol CYP, respectively. No evidence of Compound 1 metabolism by CYP2B6, CYP2C8, CYP2C9 and CYP2C19 was observed.

The predicted contribution of each CYP on Compound 1 Form A metabolism was assessed using the relative activity of factors method. These calculations indicated that CYP1A2, CYP3A4, and CYP2D6 were responsible for 43.1, 38.9 and 18% of total metabolism, respectively.

### In Vitro CYP Inhibition of CYP1A2, CYP2C8, CYP2C9, CYP2C19, CYP2D6 and CYP3A4 in Human Liver Microsomes

This study assessed the potential inhibitory effect of Compound 1 Form A toward CYP1A2, CYP2C8, CYP2C9, CYP2C19, CYP2D6 and CYP3A4. Known inhibitors of each CYP were used as positive controls.

The results showed that Compound 1 Form A inhibited several CYP enzymes, with an IC50 for CYP1A2, CYP2C19, CYP2C9, CYP2C8 and CYP3A4 (atorvastatin only) with IC50 of 1.95, 6.17, 12.4, 52.1 and 55.7 UM, respectively.

There was no inhibition of CYP2D6 or CYP3A4 using midazolam or nifedipine as substrate.

### In Vitro Metabolite Dependent Inhibition of CYP2D6 and CYP3A4 Study in Human Liver Microsomes

This study assessed the metabolism dependent inhibition potential of Compound 1 Form A toward CYP3A4 and CYP2D6 in human liver microsomes. Troleandomycin and paroxetine, known metabolism dependent inhibitors of CYP3A4 and CYP2D6, respectively were used as positive controls.

The overall results showed that Compound 1 Form A is not a metabolism dependent inhibitor of CYP3A4 and CYP2D6.

### PXR and AhR Receptor Activation by Compound 1 Form A

The objective of this study was to determine the human and rat pregnane X receptor (PXR) and human and rat aryl hydrocarbon receptor (AhR) receptor activation potential of Compound 1 Form A. The activation of PXR is a marker for induction of CYP3A4/5 and CYP2C6 and the activation of AhR is a marker for induction of CYP1A.

Compound 1 activated the PXR receptor with a mean maximum fold activation of 4.58 in human (29.6% of the positive control) with a calculated F2 value of 5.05 UM. Mean activation was 4.70 fold in rat at 30 μM.

Compound 1 activated the AhR receptor with a mean maximum fold activation of 1.78 in human (1.11% of the positive control) and 1.42 in rat (0.946% of the positive control) at 30 μM. This suggests that Compound 1 is a potential inducer of CYP3A4 and CYP2C6.

### Example 5. In Vivo Studies

Compound 1 (e.g., Form A) prevented Alzheimer's triggered aberrant synaptic signaling while preserving physiological Glu activation. Studies have been conducted to show oral Compound 1 effectively occupied brain mGluR5 sites visualized by [¹⁸F]FPEB PET at doses 250-fold below adverse effect levels in rodent and non-human primate. For aged transgenic and double knock-in mouse Alzheimer's models, SV2A PET imaging with [¹⁸F]SynVesT-1 revealed cortical and hippocampal synapse density decreases, which were fully recovered by treatment with Compound 1. The disease-modifying benefit persists after drug washout. Tau accumulation in double knock-in mice was also reduced by Compound 1 treatment. Single nuclei transcriptomics demonstrated that Compound 1 treatment normalized expression patterns to a much greater extent in neurons than glia. Production of the microglial mediator, C1q, was not altered by Compound 1, but Alzheimer-gene-dependent C1q localization to synapses and synaptic engulfment were prevented. Thus, selective modulation of mGluR5 reversed neuronal gene expression changes to protect synapses from damage by microglial mediators.

### [¹⁸F]FPEB Displacement Studies

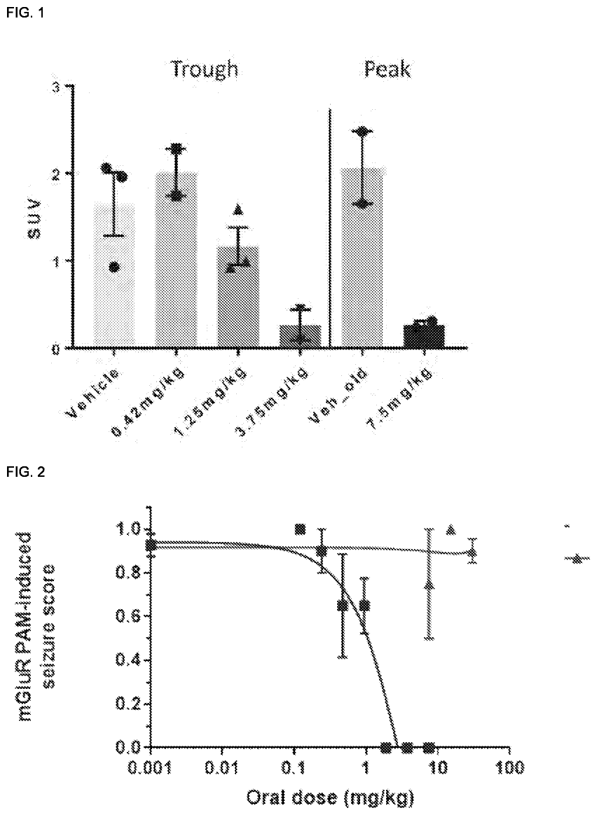

A mGluR5 PET ligand, [¹⁸F]FPEB, was used to assess receptor occupancy in mice. Mice were dosed orally twice a day with different doses of Compound 1. The available mGluR5 sites in brain were assessed in anesthetized mice both at peak 1 h after a single dose, and at trough after 7 days of twice daily (b.i.d.) oral dosing. The 3.75 mg/kg b.i.d. dose maintains >90% receptor occupancy throughout the dosing period. This is consistent with the free trough drug level of 15 nM and the inhibitor constant (Kᵢ) of Compound 1 for [³H]-MPEPy displacement from mGluR5 being 0.6 nM. The 3.75 mg/kg b.i.d. dosing regimen maintains free drug 25-fold above the Kᵢ. As shown in FIG. 1, Compound 1 is able to block the uptake of [¹⁸F] FPEB into the brain of mice. Displacement studies were conducted at expected trough levels and conducted at three dose levels: 0.42, 1.25 and 3.75 mg/kg. The 3.75 mg/kg dose was able to achieve >90% receptor occupancy at trough.

PET Imaging and Immunohistochemistry Study in APP/PS1 Mouse Model with Washout Period

The objective of this study was to assess synaptic density of control and APP/PS1 mice before and after treatment with Compound 1, and to assess the reversibility of any observed effects following a drug washout period.

Vehicle (95% polyethylene glycol (PEG) 400/5% solutol) or Compound 1 at 7.5 mg/kg/day (3.75 mg/kg q12) was given to control and APP/PS1 mice for at least 28 days by oral administration. A subset of mice stopped treatment after four weeks, then underwent a four-week washout period.

To further maximize translational relevance, in other studies mice were aged to the point that synapse loss was detectable using SV2A PET. At 12 months of age, synapse density, as measured by hippocampal/brainstem [¹⁸F]SDM-8 SUVR-1, is reduced in APP/PS1 mice relative to wild-type. Critically, rescans of the same mice after a one-month treatment course with Compound 1 showed a highly significant increase in synaptic density to a level matching wild-type mice.

Reduced synaptic density in APP/PS1 samples relative to controls was confirmed by immunohistochemical staining of pre- and post-synaptic markers (SV2A and PSD-95) in hippocampal and cortical sections. Compound 1-treated APP/PS1 samples exhibited significant increases (relative to vehicle-treated samples) with both markers and in both regions; the observed rescue of synaptic density was preserved following a one month washout period.

### SV2A PET Imaging and Immunohistochemistry Study in APP^{NLGF}/MAPT Mouse Model

The objective of this study was to assess synaptic density of control and Amyloid Precursor Protein carrying NL-G-F mutations/Microtubule-associated protein tau (APP^{NLGF}/MAPT) (dKI—double knock-in) mice before and after treatment with Compound 1. Vehicle (95% PEG-400/5% Solutol) or Compound 1 at 7.5 mg/kg/day (3.75 mg/kg q12) was given to control and dKI mice for at least 28 days by oral administration.

To further maximize translational relevance, in other studies mice were aged to the point that synapse loss was detectable using SV2A PET. At 12 months of age, synapse density, as measured by hippocampal/brainstem [¹⁸F]SDM-8 SUVR-1, was reduced in dKI mice relative to wild-type. Critically, rescans of the same mice after a one-month treatment course with Compound 1 showed a highly significant increase in synaptic density.

Reduced synaptic density in dKI samples relative to controls was confirmed by immunohistochemical staining of pre- and post-synaptic markers (SV2A and PSD-95) in hippocampal and cortical sections. Compound 1-treated dKI samples exhibited significant increases (relative to vehicle-treated samples) of SV2A in hippocampus and of both markers in the cerebral cortex.

### Prevention of Positive Allosteric Modulator Induced Seizures

The objective of this study was to assess the enantiomeric selectivity of Compound 1 for the mGluR5 receptor as a function of seizure prevention. It had been previously reported that PAMs of mGluR5 are capable of inducing seizures in C57/BI6J mice. It has also been shown previously that Compound 1 at a single 7.5 mg/kg dose prevents seizures induced by (4R,5R)-5-(4-Fluorophenyl)-4-(5-((5-fluoropyridin-3-yl)ethynyl)pyridin-3-yl)oxazolidin2-one (PAM).

Compound 1 (0.12, 0.24, 0.47, 0.94, 1.88, 3.75 or 7.5 mg/kg), enantiomer of Compound 1 (7.5, 15 or 30 mg/kg) or vehicle were administered by oral gavage to C57/BI6J mice of 3-11 months of age. After 2 hours, a PAM compound was administered at 20 mg/kg via intraperitoneal injection. Animals were then placed in a 10 inch diameter acrylic cage for observation and video recording for 2 h post dose and seizures were scored using the Racine seizure scoring criteria and normalized to a scale of 0-1.

Compound 1 displayed dose dependent prevention of mGluR5 PAM induced seizure activity in C57/BI6J mice. Compound 1 (SAM in FIG. 2) inhibited PAM-induced seizures with an IC50 of 1.09 mg/kg (FIG. 2). Pre-treatment with high doses of the enantiomer of Compound 1 (eSAM in FIG. 2) showed no seizure prevention activity.

Seizure prevention activity of Compound 1 demonstrated dose dependence occupancy of the mGluR5 receptor in the brains of C57/BI6J mice. High doses of the enantiomer of Compound 1 showed no reduction in seizure activity, suggesting that receptor binding is stereoselective.

### Example 6. Phase I Clinical Study

### Overview of Study Design

This was an open-label, single ascending doses (SAD) study in healthy male and female participants 50 to 80 years of age (inclusive) with no history of cognitive impairment to evaluate the safety of single oral doses of Compound 1.

Six cohorts of 6 participants each received a single oral dose of Compound 1 in the fasted state at the following dose levels: 10, 40, 70, 100, 150, and 200 mg and then were followed for 7 days; the starting dose of 10 mg was <10% of the 140 mg human equivalent dose (HED) of the rat NOAEL in the GLP 28-day rat study of 15 mg/kg.

Study medication was capsules containing 5, 50, or 100 mg of nano-milled active pharmaceutical ingredient; doses for each cohort were achieved using combinations of these capsules. Doses were administered in an inpatient setting where all participants were closely monitored prior to discharge on Day 3, with follow-up in-person visits occurring on Days 4 and 7 and a phone call to inquire about general health occurring on Day 5.

Compound 1 administration was carefully monitored within and between cohorts to ensure patient safety and determination of whether to open the next dose cohort was made after all participants in a given cohort had completed the study and all available clinical and safety data were reviewed by the Investigator, Medical Monitor, DSMB, and IND Sponsor.

Following study drug administration, vital signs were monitored every 1 hour for the first 8 hours and then every 3 hours until discharge on Day 3 and an ECG was performed at approximately 6, 24, and 48 hours after drug administration. In order to monitor for changes in consciousness, the Glasgow Coma Scale (GCS) was administered pre-dose and then every 2 hours for the first 8 hours and then every 3 hours until discharge on Day 3. In order to monitor for cognitive or psychiatric side effects, the MOCA, GDS, and NPIQ were administered prior to oral dose administration, again at approximately 6 hours post-dose (approximate peak concentration), and approximately 24 hours post-dose. Adverse events were monitored continuously. Blood was collected for safety laboratory studies prior to drug dose administration and on Days 2 and 3.

### Participant Disposition, Demography and Baseline Characteristics

A total of 36 participants (6 per dose cohort) enrolled in and completed the trial. Across the cohorts, the mean age and body mass index (BMI) at baseline ranged from 68.3 to 72.8 years and from 24.8 to 27.8, respectively.

The majority of participants were white (97%) and female (58.3%), and mean scores on the cognitive and depression scales administered at baseline were not indicative of cognitive impairment or clinically meaningful signs of depression (Table 20).

TABLE 20
Participant Demography and Baseline Characteristics

| | Cohort 1 | Cohort 2 | Cohort 3 | Cohort 4 | Cohort 5 | Cohort 6 |
|---|---|---|---|---|---|---|
| Parameter | (10 mg) | (40 mg) | (70 mg) | (100 mg) | (150 mg) | (200 mg) |
| Age, Mean | 70.9 | 71.3 | 70.3 | 68.3 | 72.8 | 68.1 |
| (SD) | (3.2) | (5.6) | (2.5) | (5.5) | (6.1) | (9.1) |
| Sex | 2 | 2 | 2 | 2 | 3 | 4 |
| Male N (%) | (33.3%) | (33.3%) | (33.3%) | (33.3%) | (50.0%) | (66.7%) |
| Female N (%) | 4 | 4 | 4 | 4 | 3 | 2 |
| | (66.7%) | (66.7%) | (66.7%) | (66.7%) | (50.0%) | (33.3%) |
| Body weight | 77.6 | 77.7 | 69.0 | 71.3 | 70.6 | 72.4 |
| (kg), Mean | (13.3) | (23.5) | (7.3) | (10.8) | (9.3) | (15.4) |
| (SD) | | | | | | |
| BMI (kg/m²), | 26.5 | 27.8 | 24.8 | 26.7 | 26.0 | 24.8 |
| Mean (SD) | (4.6) | (6.2) | (3.3) | (3.5) | (2.2) | (3.5) |
| CDR, Mean | 0.0 | 0.0 | 0.0 | 0.0 | 0.0 | 0.0 |
| (SD)¹ | (0.0) | (0.0) | (0.0) | (0.0) | (0.0) | (0.0) |
| Logical | 14.2 | 13.8 | 11.2 | 11.3 | 13.2 | 13.8 |
| Memory II | (3.1) | (2.4) | (4.0) | (3.6) | (3.4) | (4.4) |
| Subscale, | | | | | | |
| Delayed, | | | | | | |
| Mean (SD)² | | | | | | |
| MMSE, Mean | 29.3 | 28.8 | 29.5 | 29.0 | 29.5 | 29.5 |
| (SD) | (0.5) | (1.5) | (0.5) | (0.6) | (0.8) | (0.8) |
| (scores >25 | | | | | | |
| are normal)³ | | | | | | |
| GCS, Mean | 15.0 | 15.0 | 15.0 | 15.0 | 15.0 | 15.0 |
| (SD)⁴ | (0.0) | (0.0) | (0.0) | (0.0) | (0.0) | (0.0) |
| GDS, Mean | 0.0 | 0.3 | 1.5 | 0.8 | 1.2 | 1.0 |
| (SD)⁵ | (0.0) | (0.8) | (1.5) | (1.0) | (1.9) | (0.9) |
| MoCA, Mean | 28.2 | 26.8 | 26.5 | 26.0 | 27.5 | 27.2 |
| (SD)⁶ | (2.1) | (2.3) | (3.4) | (2.9) | (1.4) | (1.6) |

Abbreviations: BMI = body mass index; CDR = Clinical Dementia Rating scale; GCS = Glasgow Coma Scale; GDS = Geriatric Depression Scale; MMSE = mini mental state exam; MoCA = Montreal Cognitive Assessment; SD = standard deviation.
¹Scores of above 0 indicate progressive decline due to a cognitive disorder.
²Scores range from 1 to 19; with higher scores indicating better delayed verbal recall.
³Scores range from 1 to 30; with higher scores indicating better cognitive performance
⁴Scores range from 3 to 15; with lower scores indicating impaired consciousness.
⁵Scores range from 0 to 15; with higher scores indicating a greater likelihood of depression.
⁶Scores range from 0 to 30; with higher scores indicating better cognitive performance

### Safety Results

Single oral doses of Compound 1 were well tolerated with no deaths, SAEs, or severe TEAEs observed in any participant. There was also no evidence in any participant of clinically significant changes in consciousness as measured by the GCS, or in cognitive or psychiatric side effects as measured by the MOCA, the GDS, and the NPIQ. All TEAEs were mild or moderate in intensity and 8 TEAEs were considered possibly related to treatment; all others were assessed as unlikely related. These possibly related TEAEs consisted of 3 reports of brief oral sensations (abnormal taste, tongue tingling, mouth pain), 1 brief episode of dizziness, 2 reports of transient headache (one treated with a single dose of acetaminophen 500 mg), 1 episode of transient hypertension, and 1 lab measurement of increase in triglycerides on Day 7 in a participant with a history of hypertriglyceridemia.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

What is claimed is:

1. An anhydrous (AH) crystalline form (Form A) of (4R,5R)-5-(2-chlorophenyl)-4-(5-(phenylethynyl)pyridin-3-yl)oxazolidin-2-one 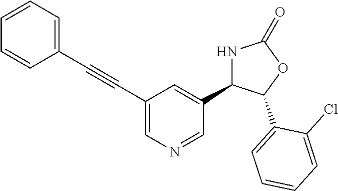 [SMILES: O=C1N[C@H](c2cncc(C#Cc3ccccc3)c2)[C@@H](c2ccccc2Cl)O1]
(Compound 1), having an XRPD pattern comprising peaks of 2θ angles at 9.02, 11.65, and 11.86 degrees 2θ, each ±0.2 degrees 2θ, as measured by X-ray diffractometry by irradiation with Cu Kα X-rays.

2. The AH crystalline form of claim 1, wherein the XRPD pattern further comprises peaks of 2θ angles at 12.15, 14.99, 28.99, and 44.03 degrees 2θ, each ±0.2 degrees 2θ, as measured by X-ray diffractometry by irradiation with Cu Kα X-rays.

3. The AH crystalline form of claim 1 or 2, wherein the XRPD pattern further comprises three peaks of 2θ angles at 21.09, 21.49, and 21.88 degrees 2θ, each ±0.2 degrees 2θ, as measured by X-ray diffractometry by irradiation with Cu Kα X-rays.

4. The AH crystalline form of any one of claims 1-3, wherein the XRPD pattern comprises peaks of 20 angles at 9.02, 11.65, 11.86, 12.15, 14.99, 21.09, 21.49, 21.88, 28.99, and 44.03 degrees 2θ, as measured by X-ray diffractometry by irradiation with Cu Kα X-rays.

5. The AH form of any one of claims 1-4, having an XRPD pattern substantially as shown in FIG. 3.

6. The AH form of any one of claims 1-5, having a melting endothermic peak at about 134° C.-138° C. in a differential scanning calorimetry (DSC) thermogram.

7. The AH form of any one of claims 1-6, having a DSC thermogram substantially as the DSC graph shown in FIG. 4.

8. The AH form of any one of claims 1-7, having a thermogravimetric analysis (TGA) weight loss of about 0.02% (w/w) between ambient and about 250° C.

9. The AH form of any one of claims 1-8, having a TGA substantially as the TGA graph shown in FIG. 4.

10. The AH form of any one of claims 1-9, which is substantially purified.

11. A method for preparing an anhydrous (AH) crystalline form of (4R,5R)-5-(2-chlorophenyl)-4-(5-(phenylethynyl)pyridin-3-yl)oxazolidin-2-one (Compound 1), comprising precipitating the anhydrous crystalline form from a solution comprising Compound 1 and a solvent selected from the group consisting of ethyl acetate (EtOAc), heptane, and mixtures thereof.

12. The method of claim 11, wherein the solvent is a mixture of EtOAc and heptane.

13. The method of claim 12, wherein the ratio of EtOAc and heptane is 90:10, 80:20, 70:30, 60:40, 50:50, 40:60, 30:70, 20:80 or 10:90.

14. The method of any one of claims 11-13, further comprising cooling the solution.

15. The method of claim 14, further comprising isolating the AH crystalline form by filtration.

16. An anhydrous (AH) crystalline form of Compound 1 prepared by the method of any one of claims 11-15.

17. A solvate form (Form B) of (4R,5R)-5-(2-chlorophenyl)-4-(5-(phenylethynyl)pyridin-3-yl)oxazolidin-2-one (Compound 1), having an XRPD pattern comprising at least three of the following peaks of 2θ angles at 16.07, 16.27, 16.58, and 16.77 degrees 2θ, each ±0.2 degrees 2θ, as measured by X-ray diffractometry by irradiation with Cu Kα X-rays.

18. The solvate Form B of claim 17, wherein the XRPD pattern further comprises three or more of the following peaks of 2θ angles at 13.18, 13.4, 13.54, 13.54, and 13.70 degrees 2θ, each ±0.2 degrees 2θ, as measured by X-ray diffractometry by irradiation with Cu Kα X-rays.

19. The solvate Form B of claim 17 or 18, wherein the XRPD pattern further comprises peaks of 20 angles at 37.31, and 39.92 degrees 2θ, each ±0.2 degrees 2θ, as measured by X-ray diffractometry by irradiation with Cu Kα X-rays.

20. The solvate Form B of any one of claims 17-19, wherein the XRPD pattern comprises peaks of 2θ angles at 13.18, 13.4, 13.54, 13.54, 13.70, 16.07, 16.27, 16.58, 16.77, 37.31, and 39.92 degrees 2θ, each ±0.2 degrees 2θ, as measured by X-ray diffractometry by irradiation with Cu Kα X-rays.

21. The solvate Form B of any one of claims 17-20, having an XRPD pattern substantially as shown in FIG. 5.

22. The solvate Form B of any one of claims 17-21, having a melting endothermic peak at about 134° C.-138° C. in a differential scanning calorimetry (DSC) thermogram.

23. The solvate Form B of any one of claims 17-22, having a DSC thermogram substantially as the DSC graph shown in FIG. 6.

24. The solvate Form B of any one of claims 17-23, having a thermogravimetric analysis (TGA weight loss of about 6.7% (w/w)) between ambient and about 200° C.

25. The solvate Form B of any one of claims 17-24, having a TGA substantially as the TGA graph shown in FIG. 6.

26. The solvate Form B of any one of claims 17-25, wherein the solvate is an isopropyl alcohol solvate.

27. The solvate Form B of any one of claims 17-26, which is substantially purified.

28. A method for preparing solvate Form B of (4R,5R)-5-(2-chlorophenyl)-4-(5-(phenylethynyl)pyridin-3-yl)oxazolidin-2-one (Compound 1), comprising the evaporation of a mixture of Compound 1 and isopropyl alcohol under inert atmosphere in a dry-box.

29. The method of claim 28, wherein the evaporation occurs over a period of 1-2 weeks.

30. The method of claim 28 or 29, further comprising drying the crystalline Form B under vacuum.

31. A crystalline Form B of Compound 1 prepared by the method of any one of claims 28-30.

32. A solvate form (Form C) of (4R,5R)-5-(2-chlorophenyl)-4-(5-(phenylethynyl)pyridin-3-yl)oxazolidin-2-one (Compound 1), having an XRPD pattern comprising only two peaks between 13.32 and 13.82 degrees 2θ, each ±0.2 degrees 2θ, as measured by X-ray diffractometry by irradiation with Cu Kα X-rays.

33. The solvate Form C of claim 32, wherein the XRPD pattern further comprises only three peaks between 32.22 and 32.96 degrees 2θ, each ±0.2 degrees 2θ, or comprises peaks of 2θ angles at 3.4, 8.33, 10.94, 16.32, and 16.66 degrees 2θ, each ±0.2 degrees 2θ, as measured by X-ray diffractometry by irradiation with Cu Kα X-rays.

34. The solvate Form C of claim 32 or 33, wherein the XRPD pattern comprises peaks of 2θ angles at 3.4, 8.33, 10.94, 13.32, 13.82, 16.32, 16.66, 32.22, 32.59, 32.96 degrees 2θ, each ±0.2 degrees 2θ, as measured by X-ray diffractometry by irradiation with Cu Kα X-rays.

35. The solvate Form C of any one of claims 32-34, having an XRPD pattern substantially as shown in FIG. 7.

36. The solvate Form C of any one of claims 32-35, having an XRPD pattern substantially as shown in FIG. 9.

37. The solvate Form C of any one of claims 32-36, having a melting endothermic peak at about 129-133° C. in a differential scanning calorimetry (DSC) thermogram.

38. The solvate Form C of any one of claims 32-37, having a DSC thermogram substantially as the DSC graph shown in FIG. 8.

39. The solvate Form C of any one of claims 32-38, having a thermogravimetric analysis (TGA) a weight loss of about 3.4% (w/w) between ambient and about 200° C.

40. The solvate Form C of any one of claims 32-39, having a TGA substantially as the TGA graph shown in FIG. 8.

41. The solvate Form C of any one of claims 32-40, wherein the solvate is an isopropyl alcohol solvate.

42. The solvate Form C of any one of claims 32-41, which is substantially purified.

43. A method for preparing solvate Form C of (4R,5R)-5-(2-chlorophenyl)-4-(5-(phenylethynyl)pyridin-3-yl)oxazolidin-2-one (Compound 1), comprising the precipitation of the solvate form from a solution comprising Compound 1 and isopropyl alcohol.

44. The method of claim 43, wherein the solution is stirred at 20° C. over two weeks.

45. The method of claim 43 or 44, further comprising isolating the crystalline Form C by filtration.

46. A crystalline Form C of Compound 1 prepared by the method of any one of claims 43-45.

47. The solvate Form C of any one of claims 32-36, having endothermic peaks at about 89.4° C. and at about 134.2° C. in a differential scanning calorimetry (DSC) thermogram.

48. The solvate Form C of any one of claim 32-36 or 47, having a DSC thermogram substantially as the DSC graph shown in FIG. 10.

49. The solvate Form C of any one of claim 32-36, 47 or 48, having a thermogravimetric analysis (TGA) weight loss of about 3.2% (w/w) between ambient and about 200° C.

50. The solvate Form C of any one of claim 32-36, or 47-49, having a TGA substantially as the TGA graph shown in FIG. 10.

51. A method for preparing solvate Form C of (4R,5R)-5-(2-chlorophenyl)-4-(5-(phenylethynyl)pyridin-3-yl)oxazolidin-2-one (Compound 1), said method comprising:
(i) dissolving Compound 1 in isopropyl alcohol to form a saturated solution;
(ii) filtering the mixture of step (i) into a new vial that is placed in an outer vial containing an anti-solvent;
(iii) stirring the inner vial mixture of step (ii) to precipitate solids; and
(iv) filtering the solids of step (iii) and drying the solids under vacuum at room temperature.

52. The method of claim 51, wherein the anti-solvent is pentane.

53. A solvate Form C of Compound 1 prepared by the method of claim 51 or 52.

54. A pharmaceutical composition comprising an effective amount of the crystalline or solvate form of any one of claim 1-10, 16-27, 31-42, 46-51, or 53.

55. The pharmaceutical composition of claim 54, comprising between about 5% (w/w) to about 30% (w/w) of the crystalline or solvate forms.

56. The pharmaceutical composition of claim 55, comprising about 5% (w/w), 10% (w/w), 24% (w/w) or about 25% (w/w) of the crystalline or solvate forms.

57. The pharmaceutical composition of any one of claims 54-56, wherein the composition is a nanomilled suspension or a spray dried nanosuspension.

58. The pharmaceutical composition of any one of claims 54-57, further comprising a pharmaceutically acceptable plasticizer, binder, bulking agent, carrier, excipient, lubricant, disintegrant, and/or surfactant.

59. The pharmaceutical composition of claim 58, further comprising at least one of hypromellose, sodium lauryl sulfate, or lactose monohydrate.

60. The pharmaceutical composition of claim 59, further comprising at least one of croscarmellose or magnesium stearate.

61. The pharmaceutical composition of any one of claims 54-60, wherein the composition is in capsule form.

62. The pharmaceutical composition of any one of claims 54-61, wherein the the capsule is selected from a hard hydroxy propyl methylcellulose capsule, hard gelatin capsule, or soft gelatin capsule.

63. The pharmaceutical composition of any one of claims 54-62 comprising the components in the following table:

| Component | Weight percentages (w/w) |
|---|---|
| Compound 1 | 5%-30% |
| Binder | 0-10% |
| Surfactant | 0-5% |
| Carrier and bulking agent | 0-95% |
| Disintegrant | 0-10% |
| Lubricant | 0-5% |

provided the amount of binder, surfactant, carrier, disintegrant, and lubricant are not all 0%.

64. The pharmaceutical composition of claim 63, wherein the binder is hypromellose, the surfactant is sodium lauryl sulfate, the carrier and binding agent is lactose monohydrate, the disintegrant is croscarmellose, and the lubricant is magnesium stearate.

65. A method of treating Alzheimer's Disease, preventing seizure, reversing synapse loss, or reducing Tau accumulation of a subject in need thereof, comprising treating the subject with a therapeutically effective amount of the crystalline or solvate form of any one of claim 1-10, 16-27, 31-42, 46-51, or 53.
